(19) United States
(12) Patent Application Publication
(10) Pub. No.: US 2025/0270511 A1
(43) Pub. Date: Aug. 28, 2025

(54) GENETICALLY ENGINEERED CELLS, THEIR USES, AND METHODS OF MAKING SAME

(71) Applicants: The United States of America, as represented by the Secretary, Department of Health and Human Servic, Bethesda, MD (US); Gemysis. Inc., Newtown, PA (US)

(72) Inventors: Rosandra N. Kaplan, Potomac, MD (US); Sabina A. Kratzmeier, Olney, MD (US); Cristina Contreras Burrola, Bethesda, MD (US); Briana C. Bergen, Newtown, PA (US); James Cronk, Baltimore, MD (US); Kailey Jackett, Syracuse, NY (US)

(73) Assignees: The United States of America, as represented by the Secretary, Department of Health and Human Servic, Bethesda, MD (US); Gemysis, Inc., Newtown, PA (US)

(21) Appl. No.: 18/858,315

(22) PCT Filed: Apr. 20, 2023
(86) PCT No.: PCT/US2023/066025

§ 371 Date: Oct. 19, 2024

Related U.S. Application Data
(60) Provisional application No. 63/333,000, filed on Apr. 20, 2022.

(51) Int. Cl. C12N 5/077; A61K 35/28; A61P 35/00; C07K 14/54; C07K 14/705; C07K 14/71; C12N 15/86

(52) U.S. Cl. CPC C12N 5/0669; A61K 35/28; A61P 35/00; C07K 14/5434; C07K 14/70503; C07K 14/70575; C07K 14/71; C12N 15/86; C12N 2510/00; C12N 2740/15043

(57) ABSTRACT

In aspects, the disclosure provides genetically engineered cells, uses of the cells, and methods of making the cells.

### CROSS-REFERENCE TO RELATED APPLICATIONS

This application claims the priority benefit of U.S. Provisional Patent Application No. 63/333,000, filed Apr. 20, 2022, the disclosure of which is incorporated herein by reference in its entirety.

### STATEMENT REGARDING FEDERALLY SPONSORED RESEARCH OR DEVELOPMENT

This invention was made with Government support under ZIABC011334-10 and ZIABC011332-06 awarded by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 727,288 Byte Extensible Markup Language (xml) file named “766891_ST26.xml,” created on Apr. 20, 2023.

### BACKGROUND

Metastasis is the primary cause of death in patients with solid tumors. A deeper understanding of the regulators of this process is needed in order to develop effective therapeutic strategies.

Harnessing the immune system to target and eliminate distant metastatic lesions is a major challenge. Most immunotherapeutic strategies, including CAR-T cell therapy, are limited by immunosuppression in the tumor and pre-metastatic tumor microenvironment. Moreover, in mammals having primary tumors, immune cell populations are dysregulated and upregulate a core immune suppression gene signature typically resulting in metastatic tumor growth at one or more sites distant from the primary tumor.

There is a desire for an effective preventative and/or treatment method for metastasis.

There is also a need to rebalance dysregulated physiological microenvironments such as dysregulated tumor, immune and neurological microenvironments to treat primary tumors, treat and prevent metastasis, treat immune diseases and disorders, and neurological diseases and disorders and improve immunotherapy when used in combination.

### BRIEF SUMMARY

In aspects, the disclosure provides a myeloid cell or mesenchymal cell comprising exogenous mRNA, wherein the exogenous mRNA encodes IL12, membrane tethered IL12 (mIL12), a IL6 decoy receptor (IL6DR), CD40 Ligand (CD40L), a soluble Triggering Receptor expressed on Myeloid cells 2 decoy receptor (sTREM2), a tissue inhibitor of metalloproteinases (TIMPs), a dominant negative transforming growth factor β receptor II (TGFβRII), or a prostaglandin E2 receptor 2 decoy receptor (EP2DR).

In aspects, the disclosure provides a method of making a myeloid cell or mesenchymal cell that expresses a protein, the method comprising introducing to the myeloid cell or mesenchymal cell an mRNA (e.g., an exogenous mRNA) encoding IL12, membrane tethered IL12 (mIL12), a IL6 decoy receptor (IL6DR), CD40 Ligand (CD40L), a soluble Triggering Receptor expressed on Myeloid cells 2 decoy receptor (sTREM2), a tissue inhibitor of metalloproteinases (TIMPs), a dominant negative transforming growth factor β receptor II (TGFβRII), or a prostaglandin E2 receptor 2 decoy receptor (EP2DR).

In aspects, the disclosure provides a myeloid cell or mesenchymal cell comprising exogenous mRNA, wherein the exogenous mRNA encodes viral accessory protein x (Vpx).

In aspects, the disclosure provides a method of making a myeloid cell or mesenchymal cell that expresses a protein, the method comprising introducing to the myeloid cell or mesenchymal cell an mRNA encoding viral accessory protein x (Vpx).

In aspects, the disclosure provides a method of genetically modifying a myeloid cell or mesenchymal cell, the method comprising (a) introducing to the cell an mRNA encoding viral accessory protein x (Vpx), and (b) transducing the cell with a vector.

In aspects, the disclosure provides a myeloid cell or mesenchymal cell, wherein the cell has been genetically modified to inactivate, knockdown, or remove S100A8, S100A9, ARG1, IDO1, IL4, TGFβ1, TGFβ2, TGFβ3, ADAM17, CD39, CD73, CD274, CYBB/gp91phox/NOX2, BACE1, NCKAP1L, TREM2, EP2, IL12A, IL12B, or TNFA.

In aspects, the disclosure provides a method of making a genetically modified myeloid cell or mesenchymal cell, the method comprising using CRISPR to inactivate, knockdown, or remove S100A8, S100A9, ARG1, IDO1, IL4, TGFβ1, TGFβ2, TGFβ3, ADAM17, CD39, CD73, CD274, CYBB/gp91 phox/NOX2, BACE1, NCKAP1L, TREM2, EP2, IL12A, IL12B, or TNFA.

In aspects, the disclosure provides a nucleotide sequence for a 3′ UTR that improves the stability of a mRNA sequence. In aspects, cDNA is used to create the 3′ UTR sequence, and the cDNA comprises the sequence of KJ1 (SEQ ID NO: 6), mtRNR1-KJ1 (SEQ ID NO: 7), mtRNR1-AES-KJ1 (SEQ ID NO: 8), KJ2 (SEQ ID NO: 9), mtRNR1-KJ2 (SEQ ID NO: 10), mtRNR1-AES-KJ2 (SEQ ID NO: 11), or KJ3 (SEQ ID NO: 12).

Additional aspects are as described herein.

### BRIEF DESCRIPTION OF THE DRAWINGS

FIG. 1A presents a series of flow cytometry graphs showing expression levels of epidermal growth factor receptor (EGFR) mRNA over 20 hours in primary human monocytes.

FIG. 1B is a line graph showing expression levels of IL12 mRNA over the 20 hours after no electroporation (No EP) or electroporation (EP) with a truncated epidermal growth factor receptor and interleukin-12 (tEGFR-IL12) mRNA or no mRNA (blank EP) in fresh primary human monocytes (obtained from apheresis elutriation fraction) as measured by ELISA.

FIG. 2 is a graph showing expression levels of IL12 mRNA in primary human monocytes from Donor 1 or Donor 2 over the 6 hours after electroporation (EP) without mRNA (Blank EP) or with IL12 mRNA (IL12 EP) as measured by ELISA.

FIG. 3 presents flow cytometry plots of human Genetically Engineered Myeloid cells (GEMys) after electroporation with mRNA encoding human single chain IL12 or GFP.

FIG. 4 is a graph showing the expression levels of IL12 mRNA and IL12 protein expression in human GEMys or non-engineered myeloid cells over the 48 hours after EP with IL12 mRNA or no mRNA (Blank EP) as measured by RT-PCR and ELISA respectively.

FIG. 5A presents a flow cytometry dot plot showing baseline levels of high EGFR (EGFRhi) expression by lentiviral vector transduction in primary human myeloid cells without electroporation (No EP).

FIG. 5B presents a flow cytometry dot plot showing levels of high EGFR (EGFRhi) expression after EP with Viral protein x (Vpx) mRNA 24 hours prior to transduction with lentiviral vector containing EGFR.

FIG. 5C presents a flow cytometry plot comparing high EGFR expression from transduction with lentiviral vector containing EGFR after EP with Vpx mRNA or without mRNA (No EP).

FIG. 6 is a graph showing the expression levels of IL12 mRNA over 24 hours following EP with IL12 mRNA in fresh GEMys and in thawed GEMys that were cryopreserved immediately after EP.

FIG. 7 is a bar graph showing interferon gamma (IFNγ) production in donor matched lymphocytes after co-culture with human unstimulated T cells (Unstim), human T cells alone, No EP human monocytes (apheresis elutriation fraction that is not electroporated) and human T cells, GFP human monocytes/GEMys (genetically engineered apheresis elutriation fraction) and human T cells, human IL12 GEMys (genetically engineered myeloid cells-cells obtained from human apheresis elutriation fraction) and human T cells, No EP human monocytes (apheresis elutriation fraction without genetic engineering) alone, GFP human GEMys alone, and human IL12 GEMys alone.

FIG. 8A is a bar graph showing in vivo localization of human GEMys (genetically engineered myeloid cells) with Green Fluorescent Protein (GFP) mRNA (GFP GEMys) or human IL12 mRNA (IL12 GEMys) in the spleen, bone marrow, lung, and liver of NSG-SGM3 mice as measured by the percent of human CD45 positive cells of all live cells collected from these tissues 24 hours after intravenous GEMys injection. No cells indicates background staining levels in the tissues of mice that did not receive GEMys.

FIG. 8B is a bar graph showing the phenotype of human mRNA-GEMys in the lungs of NSG-SGM3 mice treated with GFP mRNA GEMys or IL12 mRNA GEMys as measured by CD11b⁺, CD33⁺, CD14⁺, CD15⁺, and HLA-DR⁺ cells as a percent of human CD45 positive cells in the lung.

FIGS. 9A-9E present graphs showing the expression levels of murine IL12 mRNA in the plasma (FIG. 9A), lung (FIG. 9B), spleen (FIG. 9C), liver (FIG. 9D), and orthotopic rhabdomyosarcoma tumor (FIG. 9E) of C57/B16 mice one day after no treatment, or injection with Clylcophosphamide and Fludrabine (Cy/Flu) alone, or injection with Cyclophosphamide & Fludarabine (Cy/Flu) and either fresh or cryopreserved thymocyte differentiation antigen 1.1 (Thy1.1) and IL12 mRNA murine GEMys.

FIG. 10A is a flow cytometry plot showing expression levels of CD40L in murine GEMys that are untransduced (UTD), or transduced with a lentiviral vector encoding either Thy1.1 or cluster of differentiation 40 ligand (CD40L).

FIG. 10B is a flow cytometry dot plot showing expression levels of CD40L in murine GEMys transduced with CD40L lentivirus.

FIG. 11 is a graph showing CD40 signaling in the human embryonic kidney (HEK)-Blue CD40L reporter cell line which allow for detection of bioactive CD40L through the activation of nuclear factor kappa-light-chain-enhancer of activated B cells (NF-κB) following CD40 stimulation. HEK-Blue CD40L reporter cell line following co-culture with untransduced, Thy1.1, or CD40L GEMys using NF-κB induction as measured by optical density (OD) at 650 nm. The dotted line shows the baseline OD from the HEK-Blue CD40L reporter cell line.

FIG. 12A presents bar graphs showing expression levels of cluster of differentiation 80 (CD80), cluster of differentiation 86 (CD86), and major histocompatibility complex II (MHCII) in classical denedritic cells (cDCs) after co-culture with untransduced, Thy1.1, and CD40L GEMys.

FIG. 12B presents bar graphs showing expression levels of CD80, CD86, and MHCII in B cells after co-culture with untransduced, Thy1.1, and CD40L GEMys.

FIG. 13 is an illustration showing the proposed mechanism of action of soluble triggering receptor expressed on myeloid cells 2 (sTREM2)-GEMys.

FIG. 14 is a bar graph showing the expression levels of human sTREM2 in murine GEMys that are untransduced (UTD) or transduced with a lentiviral vector encoding human STREM2 as measured by ELISA.

FIG. 15 is a graph showing the probability of survival over the 90 days following injection of F4 murine sygeneic osteosarcoma tumor cells in C57BL/6 mice after treatment with cyclophosphamide and fludarabine (Cy/Flu) alone or in combination with murine GEMys expressing soluble TREM2.

FIG. 16A is a drawing showing the experimental design used to testing the efficacy of sTREM2 GEMy and GD2 chimeric antigen receptor T cells (CART) treatments on tumor growth in NSG mice bearing midline glioma, also known as diffuse intrinsic pontine glioma (DIPG).

FIG. 16B is a graph showing the tumor growth curves of NSG mice bearing midline glioma also known as diffuse intrinsic pontine glioma (DIPG) when given a mock treatment or treated with: sTREM2 GEMy alone at Day 0, GD2 CART alone at Day 7, a combination of STREM2 GEMys with subtherapeutic dosing of GD2 CART given together at Day 7, or a combination of sTREM2 GEMys given at Day 0 and GD2 CART given at Day 7. The dashed line represent when no more tumor is measurable.

FIG. 16C shows a schematic of mechanisms by which sTREM2 in combination with immunotherapy can alleviate myeloid mediated immune suppression and improve anti-tumor T cell efficacy.

FIG. 17 is a bar graph showing the percent of GFP positive cells in the SC human monocyte cell line (CRL-9855) that was untransduced (UTD) or transduced with a lentiviral vector encoding GFP and either the promoter EF1a or the myeloid specific promoters sp107, sp144, MMP12, combined sp107 and MMP14, or combined sp144 and MMP14 promoter sequences.

FIG. 18 is a bar graph showing the GFP positive percentage of CD14+ cells in primary human monocytes cultures that underwent EP of Vpx mRNA 24 hours prior to treatment and were untransduced or transduced with a lentiviral vector encoding GFP and either the promoter EF1a or the myeloid specific promoters, sp107, sp144, MMP14, combined sp107 and MMP14, or combined sp144 and MMP14 promoter sequences.

### DETAILED DESCRIPTION

It has been found that introduction of mRNA into myeloid cells, e.g., human elutriated monocytes (RO elutration fraction from apheresis cell product), allows for fast and transient expression of cargo proteins without the need for extended culture protocols, which are technically challenging given the short lifespan of these types of cells. Myeloid cells include macrophages, monocytes, dendritic cells, monocytic dendritic cells, and granulocytes.

Provided herein are synthetic messenger ribonucleic acids (mRNAs) for transducing, e.g., autologous or allogenic myeloid cells for use in rebalancing dysregulated niches or microenvironments.

Also provided herein are methods for advantageously using synthetic messenger ribonucleic acid (mRNA) to transduce, e.g., myeloid cells, and using such myeloid cells to rebalance dysregulated niches or microenvironments, such as tumor, immune, and neurological microenvironments.

In aspects, the disclosure provides a genetically engineered myeloid cell (GEMy) comprising mRNA (e.g., exogenous mRNA) introduced into the myeloid cell, such as by electroporation or other suitable method for introducing a desired mRNA into the myeloid cell. Provided herein are myeloid cells transduced with one or more mRNAs (e.g., exogenous mRNA) for rebalancing dysregulated niches. In aspects the mRNA is modified to include sequences that specifically promote stability in myeloid cells. In aspects, the dysregulated niche is a tumor microenvironment (TME) or metastatic microenvironment at a site distant from the site of a primary tumor.

In aspects, the disclosure provides a composition comprising (a) myeloid cells, including but not limited to monocytes, monocytic dendritic cells, macrophages, and neutrophils; or (b) genetically modified hematopoietic stem and progenitor cells (HSPCs); or (c) genetically modified mesenchymal cells; or (d) any combination of (a), (b), and (c), wherein the cells contain an mRNA (e.g., an exogenous mRNA) for expression of one or more proteins to treat or prevent a tumor and/or metastasis by modulating the tumor microenvironment.

In aspects, the disclosure provides a composition comprising (a) myeloid cells, including but not limited to monocytes, monocytic dendritic cells, macrophages, and neutrophils; or (b) genetically modified hematopoietic stem and progenitor cells (HSPCs); or (c) genetically modified mesenchymal cells; or (d) any combination of (a), (b), and (c), wherein the cells contain a modified mRNA (e.g., an exogenous modified mRNA) for expression of one or more proteins to treat an autoimmune or neurological disease or disorder by modulating a dysregulated niche. Dysregulated niches are characterized by immune suppressive myeloid cells or overactive inflammatory myeloid cells, activated pericytes, fibroblasts and active extracellular matrix remodeling and in some cases activated, dysfunctional exhausted T cells, and overactive or absent T regulatory cells.

In aspects, any of the myeloid cells, methods for making myeloid cells, or methods for making genetically modified myeloid cells may substitute hematopoietic stem and progenitor cells (HSPCs) or genetically modified mesenchymal cells (GEMesys) for said myeloid cells.

In aspects, the disclosure provides a myeloid cell or mesenchymal cell comprising lentivirus or mRNA (e.g., exogenous mRNA), wherein the lentivirus or mRNA encodes IL12, a IL6 decoy receptor (IL6DR), CD40 Ligand (CD40L), a soluble Triggering Receptor expressed on Myeloid cells 2 decoy receptor (sTREM2), a tissue inhibitor of metalloproteinases (TIMPs), a dominant negative transforming growth factor β receptor II or III (TGFβRII or TGFβRIII), or an EP2 decoy receptor. In aspects, the disclosure provides a myeloid cell or mesenchymal cell comprising exogenous mRNA, wherein the exogenous mRNA encodes viral accessory protein x (Vpx).

mRNA is not constrained by size limits; and it does not integrate into the host genome.

Provided herein are synthetic mRNAs for use in transfecting the, e.g., myeloid cells wherein the mRNA comprises an open reading frame (ORF) encoding a protein for treating a dysregulated microenvironment. In aspects, the mRNA further comprises 5′ and 3′ untranslated regions (UTRs), a 5′ cap and 3′ poly(A) tail. In aspects, the mRNA further comprises an initiation of translation sequence, such as, a Kozak sequence GCC (GCCRCCATGG) (SEQ ID NO: 1) where R is a purine (A or G) (Kozak, Gene 299:1-34 2002). In aspects the sequence comprises GCCGCCACC before the start codon of the mRNA. In aspects, the mRNA further comprises modifications of the 3′ UTR to improve myeloid cell or mesenchymal cell specific stability and translation into protein.

In aspects, the mRNA comprises two sequences encoding subunits of a therapeutic protein joined by a linker, for example, an mRNA comprising a polynucleotide sequence encoding an IL-12 p40 subunit, a linker, and an IL-12 p35 subunit. Suitable linkers for use in the polynucleotides include glycine-serine (GS) linkers, such as, Gly6Ser and (Gly4Ser) n linkers wherein n is the number of repeats of the motif (n=1, 2, 3, 4, 5, etc.), for example, (Gly4Ser) 3.

The mRNA can be synthesized by any suitable method such as, for example, by polymerase chain reaction (PCR) amplification, linearized plasmid DNA in combination with cell-free in vitro transcription (IVT) of mRNA, transcribing RNA from double stranded deoxyribonucleic acid (dsDNA, e.g., cDNA), or solid-phase synthesis of RNA.

In aspects, the DNA for synthesizing mRNA comprises an RNA promoter such as a DNA dependent RNA polymerase promoter sequence, a 5′ untranslated region (UTR), a Kozak sequence, a nucleic acid sequence encoding a protein or fusion protein for rebalancing a dysregulated microenvironment, and a 3′ UTR

In aspects, the mRNA is modified to enhance its stability, translatability, and regulatability, thereby regulating the amount and the timing of protein expression from the mRNA. In aspects, the mRNA includes co-transcriptional or post-transcriptional modifications such as but not limited to, adding a 5′ CAP and a 3′ poly-adenosine (poly A) tail. In aspects, subgenomic promoter sequences are used to generate self-amplifying RNA. In aspects, the mRNAs contain binding sequences for myeloid-specific RNA binding proteins wherein the myeloid specific binding sequences (also referred to herein as myeloid specific binding sites) are modified to modulate mRNA stability and translatability In aspects inducible mRNA transcripts may be prepared, e.g., that allow for small molecule inducible regulation (expression “on” or “off” in response to treatment) of specific proteins from these regulatable modified mRNAs. In aspects the inducible gene system is a doxorubicin or FK506 binding protein 12 (FKBP) destabilizing domain or protease inducible system. In aspects canonical RNA processing mechanisms may be used, such as, e.g., capping, splicing, polyadenylation, degradation, stabilization, and trafficking. In aspects group I introns and permuted intron-exon sequences may be added to enable autocatalytic circular RNA; adding these sequences can promote circularization of RNA which promotes its stability as it is less susceptible to endonucleases.

The genetically engineered myeloid cells can be from any suitable source in a mammal, including bone marrow and blood (e.g., peripheral blood). In aspects, the genetically engineered myeloid cells are obtained by differentiating hematopoietic stem and progenitor cells (HSPCs), which can be obtained by isolating CD34⁺ cells or performing lineage depletion of bone marrow or blood cells of Ter119, CD31, CD3, CD19, CD56, and CD11b. In aspects, the genetically modified myeloid cells are genetically modified bone marrow-derived myeloid cells that can express CD33 and CXCR4. In aspects any of the myeloid cells and methods for producing myeloid cells or genetically modifying myeloid cells described herein can substitute HSPCs for myeloid cells.

Any suitable means for differentiating genetically modified HSPCs into myeloid cells can be used. For example, media supporting myeloid cell differentiation includes, but is not limited to, StemSpan SFEM II (StemCell Technologies), StemSpan CD34+ Expansion Supplement (StemCell Technologies), StemSpan Myeloid Expansion Supplement II (StemCell Technologies), or other suitable media with the addition of cytokines such as SCF, FLT3L, IL-6, IL-3, G-CSF, GM-CSF, or M-CSF or activation factors including but not limited to IFNα, IFNγ, IL-1α, TLR agonists, STING agonists, RGD peptides, or fibronectin, or any combination thereof. Genetically engineered myeloid cells can be generated in vitro prior to administration to a mammal. Genetically engineered myeloid cells can be further differentiated when administered in vivo.

In aspects, the genetically engineered myeloid cells or mesenchymal cells express CD33, (i.e., the GEMys are differentiated from CD34+ cells).

In aspects, the disclosure provides a myeloid cell or mesenchymal cell comprising mRNA wherein the exogenous mRNA encodes a polypeptide for modulating a tumor, metastatic, immune and/or neurological microenvironment. In aspects, the myeloid cell or mesenchymal cell comprises exogenous mRNAs encoding one or more polypeptides for modulating a microenvironment, such as, for example, an exogenous mRNA encoding a cytokine and an exogenous mRNA encoding a protein that activates T cells and/or induces interferon gamma (IFN-γ).

In aspects, the disclosure provides a method of making a myeloid cell or mesenchymal cell that expresses a protein, the method comprising introducing into the myeloid cell or mesenchymal cell a lentiviral vector or an mRNA (e.g., an exogenous mRNA) encoding IL12, a IL6 decoy receptor (IL6DR), CD40 Ligand (CD40L), a soluble Triggering Receptor expressed on Myeloid cells 2 decoy receptor (sTREM2), a tissue inhibitor of metalloproteinases (TIMPs), a dominant negative transforming growth factor β receptor II or III (TGFβRII or TGFβRIII), or an EP2 decoy receptor. In aspects, the disclosure provides a method of making a myeloid cell or mesenchymal cell that expresses an exogenous protein, the method comprising introducing to the myeloid cell or mesenchymal cell an mRNA encoding viral accessory protein x (Vpx). In aspects, the disclosure provides a method of genetically modifying a myeloid cell or mesenchymal cell, the method comprising (a) introducing to the cell an mRNA encoding viral accessory protein x (Vpx), and (b) transducing the cell with a vector.

In aspects, introduction of mRNA to a cell is through electroporation, though any suitable method may be used, including but not limited to liposome delivery or microfluidic squeezing protocols. In aspects, the mRNA is introduced into a myeloid cell or mesenchymal cell by pulsing, lipofection, or microfluidic squeezing protocols.

In aspects, the myeloid cell or mesenchymal cell comprises a vector, which may comprise a transgene. Examples of suitable vectors include plasmids (e.g., DNA plasmids), bacterial vectors (e.g., a Listeria or Salmonella vector), yeast vectors, and viral vectors. In aspects, the vector is a viral vector, such as retrovirus, poxvirus, e.g., an orthopox (e.g., vaccinia, modified vaccinia Ankara (MVA), Wyeth, NYVAC, TROYVAC, Dry-Vax, or POXVAC-TC), avipox (e,g., fowlpox, pigeonpox, or canarypox, such as ALVAC), raccoon pox, rabbit pox, capripox (e.g., goat pox or sheep pox), leporipox, or suipox (e.g., swinepox), adenovirus, adeno-associated virus, herpes virus, polio virus, alphavirus, baculorvirus, and Sindbis virus. In aspects, the vector is a lentiviral vector.

Retroviral vectors, including lentiviral vectors, are suitable delivery vehicles for the stable introduction of a variety of genes of interest into the genomic DNA of a broad range of target cells. Without being bound by theory, the ability of retroviral vectors to deliver unrearranged, single copy transgenes into cells makes retroviral vectors well suited for transferring genes into cells. Further, retroviruses enter host cells by the binding of retroviral envelope glycoproteins to specific cell surface receptors on the host cells. Consequently, pseudotyped retroviral vectors in which the encoded native envelope protein is replaced by a heterologous envelope protein that has a different cellular specificity than the native envelope protein (e.g., binds to a different cell-surface receptor as compared to the native envelope protein) also can be used.

There are many retroviruses and examples include: murine leukemia virus (MLV), lentivirus such as human immunodeficiency virus (HIV), equine infectious anaemia virus (EIAV), mouse mammary tumor virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). Other retroviruses suitable for use include, but are not limited to, Avian Leukosis Virus, Bovine Leukemia Virus, and Mink-Cell Focus-Inducing Virus. The core sequence of the retroviral vectors can be derived from a wide variety of retroviruses, including for example, B, C, and D type retroviruses, as well as spumaviruses and lentiviruses.

In aspects, the lentivirus is a human immunodeficiency virus (HIV), for example, type 1 or 2 (i.e., HIV-1 or HIV-2). Other lentivirus vectors include sheep Visna/maedi virus, feline immunodeficiency virus (FIV), bovine lentivirus, simian immunodeficiency virus (SIV), an equine infectious anemia virus (EIAV), and a caprine arthritis-encephalitis virus (CAEV).

In addition to the transgene, the vector can include an expression control sequence operatively linked to the transgene's coding sequence, such that expression of the coding sequence is achieved under conditions compatible with the expression control sequences. The expression control sequences include, but are not limited to, appropriate promoters, enhancers, transcription terminators, Kozak sequence, a start codon (i.e., ATG) in front of a protein-encoding gene, signal peptide sequence for targeting to the secretory pathway, splicing signal for introns, maintenance of the correct reading frame of that gene to permit proper translation of mRNA, and stop codons. Suitable promoters include, but are not limited to, a hVMD2 promoter, an SV40 early promoter, RSV promoter, adenovirus major late promoter, human CMV immediate early I promoter, poxvirus promoter, 30K promoter, I3 promoter, sE/L promoter, 7.5K promoter, 40K promoter, C1 promoter, and EF-1a promoter. In aspects myeloid specific or synthetic myeloid promoters can be used. Further, cell-type specific or inducible promoters can be used to limit expression to myeloid cells in the tumor microenvironment, such as myeloid specific synthetic promoters (sp-144, sp-107) and other suitable promoters including myeloid specific promoters for improved expression of desired cargo proteins in myeloid cells, such as, myeloid specific transcription factor promoters SP1, Pu. 1a, Pu. 1b, C/EBPa, AP1, AML-1, LYSMD1, CBF, BCL2, MYB, GATA, MMP14, and variants thereof.

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences.

Additionally, the vector can comprise a reporter to identify the transfection/transduction efficiency of the vector. Exemplary reporters include, but are not limited to, epidermal growth factor receptor (EGFR), Thy1.1 (CD90.1), or low-affinity human nerve growth factor receptor (LNGFR). Truncated EGFR (tEGFR) can be used as a reporter to measure transduction efficiency and as a potential safety switch to deplete transduced cells in vivo by using anti-EGFR antibody (such as Cetuximab).

The transgene can be any suitable transgene, such as transgene encoding one or more of a cytokine, a chemokine, an enzyme, a substrate, a transcription factor, a receptor decoy/dead receptor (e.g., sTREM2, IL6DR, or TNFα decoy/dead receptor), an antibody (e.g., scFv, IgG, or a bispecific or trispecific antibody for secretion, binding, and opsonizing tumor/increasing phagocytosis; or an antibody-drug that targets tumor cells, damaged neurons, or damaged, dead or dying cells), a suicide gene system, a CRISPR edited gene, or a protein induced after binding a receptor.

In aspects, the transgene encodes an enzyme that is an extracellular matrix remodeling protein, such as hyaluronidase. In aspects, the transgene encodes a suicide gene system, for example, a Herpes Simplex Virus Thymidine Kinase (HSVTK)/Ganciclovir (GCV) suicide gene system and an inducible Caspase suicide gene system. In aspects the transgene encodes a gene of a inducible gene system, wherein the inducible gene system is a doxorubicin or FK506 binding protein 12 (FKBP) destabilizing domain or protease inducible system. In aspects, the transgene can encode a cytokine, chemokine, or a related protein, such as IL-12, CXCL9, CXCL10 (anti-tumor); IL-10, SMAD (immune suppressing to rebalance the immune milieu), TGFβIL-2, TREM1, sTREM2, CD2AP, GPR32, FPR2, P2ry2, P2ry6, ChemR23, ERV, GPR32, GPR18, GPR37, and LGR6. In aspects, the transgene encodes IL-12, CXCL9, IL-10, STREM2, or CD2AP.

In aspects, there are one or more (e.g., 2, 3, 4, or more) transgenes, which may or may not perform complementary functions. For example, CXCL9 recruits T-cells and IL-12 activates T cells for the purpose of treating and/or preventing tumor metastasis. The one or more transgenes can be present in a single vector. Alternatively, one or more vectors can be employed each containing one or more transgenes, wherein the transgenes in the one or more vectors can be the same or different.

The disclosure also contemplates an inducible transgene. For example, in an inducible system, the expression of one or more transgenes can depend on temperature, pH, oxygen levels, and/or the presence of a particular small molecule. An inducible system can be used to target specific cells or tissues in a mammal and/or target particular disorders.

In aspects, the transgene encodes a protein that only is released after exposure to a specific extracellular matrix protein or in response to a tumor specific protein or in response to a particular secreted protein, pH change, oxygen level, or receptor signaling.

In aspects, the disclosure provides a myeloid cell or mesenchymal cell, wherein the cell has been genetically modified to remove/knockout, silence or knockdown, one or more of the following genes: S100A8, S100A9, ARG1, IDO1, IL4, TGFβ1, ADAM17, MMP9, CD39, CD73, CD274, CYBB/gp91phox/NOX2, BACE1, NCKAP1L, TREM2, EP2, IL12A, IL12B, and TNFA. In aspects, the disclosure provides a method of making a genetically modified myeloid cell or mesenchymal cell, the method comprising removing, inactivating, or editing a gene, e.g., any of S100A8, S100A9, ARG1, IDO1, IL4, TGFβ1, ADAM17, MM9, CD39, CD73, CD274, CYBB/gp91phox/NOX2, BACE1, NCKAP1L, TREM2, EP2, IL12A, IL12B, and TNFA, or others, using CRISPR.

In solid tumors, immunotherapeutic strategies have been limited by the ability of T cells to penetrate deep into tumors and to persist, due to suppression by myeloid cells accumulating in tumor microenvironment (TME). Myeloid cells are part of the innate immune system and are multifunctional. They can be directly cytotoxic, capable of antigen presentation and efficient at tissue repair. During repair, myeloid cells dampen immune response to prevent immune-mediated tissue damage. Myeloid cells are also the first responders to damaged tissue sites and the most abundant cell in the tumor microenvironment. These myeloid can be overactive and elicit continued inflammatory activation in other dysregulated microenvironments. By modulating myeloid cells as described in several aspect this approach can be used to limit myeloid mediated immune suppression to promote effective anti-tumor immunity and improve existing immunotherapy. In aspects modulating myeloid cells can allow for myeloid mediated immune suppression when the environment is highly activated, inflammatory, and induced to on-going overzealous immune activation.

In aspects, the disclosure provides a method of treating a mammal having a tumor, the method comprising administering to the mammal a therapeutically effective amount of a population of myeloid cell or mesenchymal cells as described herein.

The genetically engineered myeloid cells or mesenchymal cells can be administered to a mammal with cancer in order to treat cancer. Non-limiting examples of specific types of cancers include cancer of the head and neck, eye, skin, mouth, throat, esophagus, chest, bone, lung, colon, sigmoid, rectum, stomach, prostate, breast, ovaries, kidney, liver, pancreas, brain, intestine, heart or adrenals. More particularly, cancers include solid tumor, sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovial sarcoma, medullary thyroid carcinoma, adrenocortical carcinoma, desmoplastic small round cell tumor (DSRCT), malignant peripheral nerve sheath tumors (MPNST), pericytoma, NTRK+ and NTRK− fusion tumors, rhabdoid tumors, Fusion negative, Ewings like sarcomas, mesothelioma, Ewings tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, retinoblastoma, a blood-born tumor, acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acute lymphocyctic leukemia, acute undifferentiated leukemia, chronic myelocytic leukemia, chronic lymphocytic leukemia, hairy cell leukemia, or multiple myeloma as well as ultra/very rare cancers such as globus tumors, PECOMAs, IMF, GIST, chordomas, etc.

Treatment of cancer comprises, but is not limited to, destroying tumor cells, reducing tumor burden, inhibiting tumor growth, reducing the size of the primary tumor, reducing the number of metastatic lesions, increasing survival of the mammal, delaying, inhibiting, arresting or preventing the onset or development of metastatic cancer (such as by delaying, inhibiting, arresting or preventing the onset of development of tumor migration and/or tumor invasion of tissues outside of primary cancer and/or other processes associated with metastatic progression of cancer), delaying or arresting primary cancer progression, improving immune responses against the tumor, improving long term memory immune responses against tumor antigens, and/or improving the general health of the patient with illness. It will be appreciated that tumor cell death can occur without a substantial decrease in tumor size due to, for instance, the presence of supporting cells, vascularization, fibrous matrices, etc. Accordingly, while reduction in tumor size is preferred, it is not required in the treatment of cancer.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, e.g., cancer, or a symptom or condition thereof or preventing the recurrence of the disease, e.g., cancer.

Accordingly, the disclosure provides methods of reducing tumor growth or reducing or preventing recurrence of tumor growth in a mammal with cancer, extending survival time of a mammal with cancer, and preventing tumor dormancy in a mammal with cancer by administering the genetically engineered myeloid cells or mesenchymal cells to the mammal. In aspects, the cancer or tumor has not yet metastasized in the mammal.

Additionally, the disclosure provides a method of reducing or preventing metastasis in a mammal with cancer comprising administering the genetically engineered myeloid cells or mesenchymal cells to the mammal.

When the mammal has already been diagnosed with cancer (e.g., metastatic cancer), the genetically engineered myeloid cells or mesenchymal cells can be administered in conjunction with other therapeutic treatments such as chemotherapy, surgical resection of a tumor, treatment with targeted cancer therapy, allogeneic or autologous stem cell transplantation, T cell adoptive transfer, other immunotherapies, radiation and/or myeloid-targeting pre-conditioning regimens such as clodronate or CSF1R inhibitors. In particular, the genetically engineered myeloid cells or mesenchymal cells can be administered (concurrently or concomitantly or sequentially) with an additional therapeutic agent including, but not limited to, chimeric antigen receptor (CAR)-modified T cells, T cell receptor (TCR)-modified T cells, a dendritic cell vaccine, an oncolytic virus, chemotherapy, a small molecule, a monoclonal antibody or antigen binding fragments thereof, hormone-blocking therapy, and/or radiation therapy.

In aspects, the method includes different temporal administrations. For example, a first population of genetically engineered myeloid cells or mesenchymal cells is administered prior to (i.e., sequential delivery) a second population of genetically engineered myeloid cells or mesenchymal cells. In aspects, a first population of genetically engineered myeloid cells or mesenchymal cells is administered at the same time as a second population of genetically engineered myeloid cells or mesenchymal cells.

Genetically engineered myeloid cells (GEMys) or mesenchymal cells (GEMesys) allow for repeat dosing and combinatorial targets with multiple mRNA transcripts. Alterations to mRNA can be made to modulate its immunogenicity, stability, and the efficacy by which it is translated into protein. Different modified RNAs can be introduced to express protein products of interest to deliver locally to the tumor and metastatic microenvironment.

Genetically engineered myeloid cells or mesenchymal cells created with two or more different mRVAs introduced into the same myeloid cell or mesenchymal cells, two or more different mRVA engineered g myeloid cells or mesenchymal cells, or multiple different mRNA genetically engineered myeloid cells or mesenchymal cells or lentiviral vector-engineered myeloid cells or mesenchymal cells can be given together as a cocktail cell therapy. This would allow for multiple synergistic approaches, such as, e.g., production of the cytokine IL-12, a STREM2 decoy receptor and CD40L GEMys or tumor antigens given together to target and activate the immune milieu in the tumor microenvironment (TME). In some aspects delivery of STREM2 decoy receptor genetically engineered myeloid or mesenchymal cells can be given with IL12 genetically engineered myeloid or mesenchymal cells or with mesenchymal cells engineered to express hyaluronindase. In some aspects, CD40 expressing genetically engineered myeloid cells or mesenchymal cells and CD40L expressing genetically engineered myeloid cells or mesenchymal cells and CD40L secreting genetically engineered myeloid cells or mesenchymal cells can be can be administered as a cocktail to stimulate adaptive anti-tumor immunity and establish good prognostic tertiary lymphoid structures. This approach can also be used to tailor treatment for tumor metastases. This approach can also be combined with checkpoint blockade therapy or other immunotherapy or chemotherapy or radiotherapy approaches.

Most T cell therapies currently used in the clinic are given following a pre-conditioning regimen of cyclophosphamide and fludarabine (Cy/Flu). Therefore, the method of the disclosure can include the administration of cyclophosphamide, fludarabine, myeloid-targeting pre-conditioning including but not limited to clodronate liposomes or CSF1R inhibitors, or a combination thereof.

In aspects, the method of treating cancer includes surgical resection of a tumor and administration of the genetically engineered myeloid cells or mesenchymal cells.

The disclosure also provides a method of treating a neurodegenerative condition, autoimmune disorder, or inflammatory disorder in a mammal comprising administering the genetically engineered myeloid cells or mesenchymal cells. Exemplary neurodegenerative conditions, autoimmune disorders, and inflammatory disorders include, but are not limited to. Alzheimer's disease, amyotrophic lateral sclerosis, inflammatory bowel disease (IBD), Crohn's disease, rheumatoid arthritis, graft versus host disease (GVHD), multiple sclerosis, and alopecia areata.

Additionally, the disclosure provides a method of rebalancing dysregulated niches; restoring gut function, memory, behavior, hair growth, nail growth, and/or marrow function; or reducing or preventing movement disorders, memory dysfunction, confusion, or motility abnormalities in a mammal comprising administering the genetically engineered myeloid cells or mesenchymal cells.

The genetically engineered myeloid cells or mesenchymal cells can be administered to a mammal by various routes including, but not limited to, subcutaneous, intramuscular, intradermal, intraperitoneal, intrathecal, intravenous, intracerebroventricular, and intratumoral. In aspects, the genetically engineered myeloid cells or mesenchymal cells can be directly administered (e.g., locally administered) by direct injection into the cancerous lesion or tumor. When multiple administrations are given, the administrations can be at one or more sites in a host and a single dose can be administered by dividing the single dose into equal portions for administration at one, two, three, four or more sites on the mammal.

Preferably, the cells are administered by injection, e.g., intravenously or intraperitoneally. The pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In aspects, the pharmaceutically acceptable carrier is supplemented with human serum albumen.

In aspects the disclosure provides a method of treatment that includes administering a genetically engineered myeloid cells or mesenchymal cells that have been cryopreserved and thawed. Any suitable means of cryopreserving and thawing the genetically engineered myeloid cells or mesenchymal cell products can be used. In aspects cells can be cryopreserved in a 1:1 solution of Plasma-Lyte and CryoStor® CS10 freezing media immediately following mRNA electroporation or other modification with a vector. In aspects the cryopreservation solution can be Bambanker® or Stem-Cellbanker®

The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

The International Pat. App. No. PCT/US2020/017515 and its publication as WO 2020/163868 are incorporated by reference herein in its entirety.

The following are exemplary GEMys.

mIL12 GEMys: IL12 is a cytokine produced by myeloid cells including macrophages, monocytes, dendritic cells that is known as T cell stimulating factor. It can help skew naïve T cells into Th1 T cells during activation. It also activates NK cells. It stimulates the production of IFNg from T cells and NK cells and reduces IL4 suppression of IFNg production. The introduction of IL. 12 tethered to the membrane (mII. 12) of myeloid cell allows for presentation of the cytokine on the cell surface to T cells expressing IL12 receptor. IL12 receptor binding to this membrane tethered IL12 GEMy (mIL12 GEMy) results in downstream signaling through Stat1 and Stat3 from JAK1, JNK1/2, Akt, Tyk2, ERK1/2 signaling which skews to an M1 phenotype and is similar to activation in response to TLR stimulation. In other aspects, the membrane tethered IL12 can include but is not limited to the membrane portion of PDL1, CD80, a subunit of the IL-26 receptor, IL20RA, IL10RB, GCSFR, GMCSFR, LRP5. The signaling domains can include but not limited to no intracellular signaling domain, JAK/Stat signaling domains, or Src and Syk/Zap70 signaling domains, TLR4 signaling domains, MyD88 signaling domain, TIRAP signaling domain, TRAM signaling domain, TRIF signaling domain, CD40 signaling domain. In aspects, mIL12 GEMys can be engineered to be inducible.

IL12 GEMys: IL12 is a cytokine produced by myeloid cells including macrophages, monocytes, dendritic cells that is known as T cell stimulating factor. It can help skew naïve T cells into Th1 T cells during activation. It also activates NK cells. It stimulates the production of IFNg from T cells and NK cells and reduces IL4 suppression of IFNg production. In other aspects, the secreted IL12 signaling domains can include but are not limited to no intracellular signaling domain, JAK/Stat signaling domains, or Src and Syk/Zap70 signaling domains, TLR4 signaling domains, MyD88 signaling domain, TIRAP signaling domain, TRAM signaling domain, TRIF signaling domain, CD40 signaling domain. In aspects, IL12 GEMys can be engineered to be inducible.

IL6DR-GEMys: Interleukin 6 (IL6) is a pro-inflammatory cytokine and an anti-inflammatory myokine. Monocytes and macrophages produce IL6 in response to pathogen-associated molecular patterns to mediate fever and acute phase response during infection. IL6 is a known growth factor for hematopoietic progenitor cells and induces neutrophil mobilization from the bone marrow. Its role in breaking tumor dormancy and its association with cancer progression in breast, prostate and other cancers is well-established. IL6 is also known to play a role in COVID19 and other viruses that induce inflammatory response associated with worse outcomes. The introduction of a IL6 decoy receptor (IL6DR)-GEMys allows for low level, localized blockade of IL6 compared to systemic monoclonal antibody treatment. A synthetic decoy receptor can offer the advantage of locally limiting IL6 in a myeloid rich environment. In aspects, the IL6 decoy receptors are modified so they cannot bind GP130 to provide a means of sequestering secreted IL6 in the microenvironment without activating cells through GP130 trans-signaling. In aspects IL6DR GEMys can be engineered to be inducible.

sTREM2 GEMys: A cargo protein with a role in both central nervous system solid tumor malignancies, as well as potentially neurodegenerative diseases, is Triggering Receptor expressed on Myeloid cells 2 (TREM2), which is a protein expressed on the cell surface of myeloid cells. TREM2 has an intracellular and an extracellular domain. The intracellular domain can signal through DAP12 and the extracellular domain can be cleaved by a disintegrin and metalloproteinase (ADAM) 10/17 that leads to release of a soluble TREM2 (sTREM2). When the extracellular domain is cleaved, the intracellular domain signals intracellularly and induces immune suppressive signals in the cell. TREM2 blockade may limit cancer progression and could inhibit Alzheimer's pathology. A soluble TREM2 receptor decoy (sTREM2) can be created that does not have the intracellular domain, which would not induce intracellular signaling and immune suppression, and is trafficked to the membrane for secretion, acting as a sink and inhibitor of TREM2 signaling in the local microenvironment. sTREM2 GEMys can inhibit TREM2 signaling and could limit cancer progression and metastatic progression and potentially improve neurodegenerative diseases. In aspects sTREM2 GEMys can improve efficacy of T cell mediated immunotherapy by limiting myeloid mediated immunosuppression of adaptive immunity. In aspects sTREM2 GEMys can be engineered to be inducible.

TIMP3-GEMys: Tissue inhibitors of metalloproteinases (TIMPs) inhibit metalloproteinases (MMPs) enzymes that can degrade extracellular matrix components. TIMPs can also inhibit a disintegrin and metalloproteinases (ADAMs). In particular, TIMP3 can inhibit both ADAM10 and ADAM17, unlike TIMP1, which only inhibits ADAM10 and not ADAM17. Since both ADAM10 and ADAM17 play roles in cleaving TREM2 to become sTREM2 and also induce myeloid specific immune suppression, sTREM2 decoy receptor-GEMys in combination with TIMP3-GEMys are contemplated to limit endogenous TREM2 cleavage by ADAM10 and ADAM17 and decrease myeloid-mediated immune suppression. In aspects TIMP3-GEMys can be engineered to be inducible.

Dominant negative TGFβRII or TGFβRIII GEMys: A dominant negative TGFβRII or TGFβRIII can act as a sink for TGFβ. TGFβ is known to inhibit T cell cytotoxicity and induce myeloid mediated immune suppression and many other pleotropic effects in the TME and in different tumor settings. Delivering a TGFβRII or TGFβRIII decoy locally via GEMys could reduce toxicity and improve efficacy by limiting TGFβ in the localized TME. In aspects a dominant negative TGFβRII or TGFβRIII GEMys can be engineered to be inducible.

EP2 agonist GEMys and EP2 decoy receptor GEMys: EP2 is a prostaglandin receptor for prostaglandin E2 (PGE2). PGE2 signaling is a major modulator of inflammation and is commonly elevated during inflammatory disease processes. PGE2 is a downstream product of the cyclooxygenase 2 (COX-2) pathway. As they age, myeloid cells demonstrate increased PGE2 synthesis, and this aging phenotype also occurs similarly in the cancer setting. Myeloid-derived suppressor cells (MDSCs), which are elevated in the cancer setting in blood and the earliest metastatic microenvironment, have marked increased PGE2 expression, leading to altered glucose metabolism and bioenergetics in these cells. EP2 decoy receptor GEMys (EP2DR-GEMys) can be developed to block PGE2 signaling in macrophages as EP2 has been shown to induce myeloid cell dysfunction, and blocking EP2 restores metabolic fitness to aging myeloid cells, MDSCs, and damaged macrophages. Targeting PGE2 signaling can reverse disease associated processes including metastatic progression, memory loss and other age- and disease-related dysfunction. Use of EP2 decoy receptor GEMys are contemplated to restore a metabolically-fit myeloid compartment for treating cancer and other diseases. Further, EP2-producing GEMys that have EP2 deleted by CRISPR may promote bone development and reduced risk of fractures in patients with osteoporosis. GEMys with EP2 inactivated by CRISPR in order to eliminate EP2 receptor signaling is contemplated to promote metabolic fitness and longevity in these therapeutic engineered myeloid cells. In aspects EP2 agonist GEMys and EP2 decoy receptor GEMys can be engineered to be inducible.

In aspects the protein encoded by the mRNAs for IL12 comprises SEQ ID NO: 59, for IL6DR comprises SEQ ID NO: 60, for sTREM2 comprises SEQ ID NO: 61, human CD40L comprises SEQ ID NO: 62, for SIV Vpx comprises SEQ ID NO: 63, for human tEGFR-IL12 comprises SEQ ID NO: 65, for human tEFGR-sTREM2 comprises SEQ ID NO: 67, for murine CD40L comprises SEQ ID NO: 69. In aspects the protein encoded by the transgene IL-1RA comprises SEQ ID NO: 70. In aspects the cDNA used to create human tEFGR-sTREM2 mRNA comprises SEQ ID NO: 66.

The table below (Table 1) presents examples of genes as described herein.

TABLE 1

| Gene | Reference |
|---|---|
| Sp107 | SEQ ID NO: 19 of U.S. Pat. No. 7,709,625 |
| Sp144 | SEQ ID NO: 20 of U.S. Pat. No. 7,709,625 |
| Gene | GenBank/Other Reference |
| IL12A | BC104984.1 |
| IL12B | BC074723.2 |
| CXCL9 | BC095396.1 |
| CXCL10 | BC010954.1 |
| IL10 | CR541993.1 |
| SMAD4 | NCBI Reference Sequence: NM_005359.6 |
| TGFB1 | NCBI Reference Sequence: NM_000660.7 |
| TGFB2 | BC096235.4 |
| TGFB3 | EF560714.1 |
| IL2 | NCBI Reference Sequence: NM_000586.4 |
| TREM1 | AY074783.1 |
| TREM2 | BC032362.1 |
| CD2AP | BC069444.1 |
| GPR32 | BC095544.1 |
| FPR2 | M88107.1 |
| P2RY2 | AY136753.1 |
| P2RY6 | AF498920.1 |
| CHEMR23 | BC106927.2 |
| ERV3-2 | CH236950.1 (Gene ID: 57612) |
| HERV-W | NCBI Reference Sequence: NM_014590.4 |
| | (Gene ID: 30816) |
| HERV-K | NCBI Reference Sequence: NC_022518.1 |
| | and GenBank: AF020092.1 |
| GPR18 | BC066927.1 |
| GPR37 | NCBI Reference Sequence: NM_005302.5 |
| LGR6 | AF190501.1 |
| Gene | Gene ID |
| S100A8 | 6279 |
| S100A9 | 6280 |
| ARG1 | 383 |
| IDO1 | 3620 |
| IL4 | 3565 |
| TGFB1 | 7040 |
| TGFB2 | 7042 |
| TGFB3 | 7043 |
| ADAM17 | 6868 |
| ENTPD1 (CD39) | 953 |
| NT5E (CD73) | 4907 |
| CYBB | 1536 |
| BACE1 | 23621 |
| NCKAP1L | 3071 |
| PTGER2 (EP2) | 5732 |

The following are certain aspects of the disclosure.

1. A myeloid cell or mesenchymal cell comprising exogenous mRNA, wherein the exogenous mRNA encodes IL12, membrane tethered IL12 (mIL12), a IL6 decoy receptor (IL6DR), CD40 Ligand (CD40L), a soluble Triggering Receptor expressed on Myeloid cells 2 decoy receptor (sTREM2), a tissue inhibitor of metalloproteinases (TIMPs), a dominant negative transforming growth factor β receptor II (TGFβRII), a dominant negative transforming growth factor β receptor III (TGFβRIII), or a prostaglandin E2 receptor 2 decoy receptor (EP2DR).

2. The myeloid cell or mesenchymal cell of aspect 1, wherein the cell is a myeloid cell and the myeloid cell is differentiated from a hematopoietic stem and progenitor cell (HSPC).

3. The myeloid cell or mesenchymal cell of aspect 1, wherein the cell is a myeloid cell and the myeloid cell is a genetically modified CD34+ bone marrow-derived CXCR4+ myeloid cell.

4. The myeloid cell or mesenchymal cell of any one of aspects 1-3, wherein cDNA is used to create the exogenous mRNA, the cDNA encoding IL12 comprising the sequence of SEQ ID NO: 2, IL6DR comprising the sequence of SEQ ID NO: 3, CD40L comprising the sequence of SEQ ID NO: 4, and sTREM2 comprising the sequence of SEQ ID NO: 5.

5. The myeloid cell or mesenchymal cell of any one of aspects 1-4, wherein the exogenous mRNA comprises a 3′ UTR and cDNA is used to create the 3′ UTR, the cDNA comprising the sequence of any one of SEQ ID NOs: 10-16.

6. The myeloid cell or mesenchymal cell of any one of aspects 1-5, wherein the myeloid cell or mesenchymal cell comprises a vector.

7. The myeloid cell or mesenchymal cell of aspect 6, wherein the vector is a plasmid vector, a yeast vector, or a viral vector.

8. The myeloid cell or mesenchymal cell of aspect 6 or 7, wherein the vector is a lentiviral vector.

9. The myeloid cell or mesenchymal cell of any one of aspects 6-8, wherein the vector comprises a transgene.

10. The myeloid cell or mesenchymal cell of aspect 9, wherein the vector comprises an expression control sequence operatively linked to the transgene.

11. The myeloid cell or mesenchymal cell of aspect 10, wherein the expression control sequence is a promoter.

12. The myeloid cell or mesenchymal cell of aspect 11, wherein the promoter is a hVMD2 promoter, SV40 early promoter, RSV promoter, adenovirus major late promoter, human CMV immediate early I promoter, poxvirus promoter, 30K promoter, I3 promoter, sE/L promoter, 7.5K promoter, 40K promoter, C1 promoter, EF-1a promoter, sp107 promoter, sp144 promoter, MMP14 promoter, sp107 and MMP14 combination promoter, or sp114 and MMP14 combination promoter.

13. The myeloid cell or mesenchymal cell of aspect 12, wherein the sp107 promoter comprises SEQ ID NO: 13, the sp 144 promoter comprises SEQ ID NO: 14, the MMP14 promoter comprises SEQ ID NO: 15, the sp107 and MMP14 combination promoter comprises SEQ ID NO: 16, and the sp114 and MMP14 combination promoter comprises SEQ ID NO: 17.

14. The myeloid cell or mesenchymal cell of any one of aspects 9-13, wherein the transgene encodes a cytokine, a chemokine, an enzyme, a substrate, a receptor decoy, an antibody, or a gene of a suicide gene system.

15. The myeloid cell or mesenchymal cell of aspect 14, wherein the transgene encodes an enzyme, wherein the enzyme is hyaluronidase.

16. The myeloid cell or mesenchymal cell of aspect 14, wherein the transgene encodes a gene of a suicide gene system, wherein the suicide gene system is a Herpes Simplex Virus Thymidine Kinase (HSVTK)/Ganciclovir (GCV) suicide gene system or an inducible Caspase suicide gene system.

17. The myeloid cell or mesenchymal cell of any one of aspects 9-13, wherein the transgene encodes IL-IRA (e.g., SEQ ID NO: 18), IL-2 (e.g., SEQ ID NO: 19), IL-10 (e.g., SEQ ID NO: 20), IL-12A (e.g., SEQ ID NO: 21), IL-12B (e.g., SEQ ID NO: 22), CXCL9 (e.g., SEQ ID NO: 23), CXCL10 (e.g., SEQ ID NO: 24), SMAD4 (e.g., SEQ ID NO: 25), TGFβ1 (e.g., SEQ ID NO: 26), TGFβ2 (e.g., SEQ ID NO: 27), TGFβ3 (e.g., SEQ ID NO: 28), TREM1 (e.g., SEQ ID NO: 29), sTREM2 (e.g., SEQ ID NO: 5), CD2AP (e.g., SEQ ID NO: 31), FPR2 (e.g., SEQ ID NO: 32), P2RY2 (e.g., SEQ ID NO: 33), P2RY6 (e.g., SEQ ID NO: 34), CHEMR23 (e.g., SEQ ID NO: 35), ERV3-2 (e.g., SEQ ID NO: 36), HERV-W (e.g., SEQ ID NO: 37), HERV-K (e.g., SEQ ID NO: 38), GPR18 (e.g., SEQ ID NO: 39), GPR32 (e.g., SEQ ID NO: 40), GPR37 (e.g., SEQ ID NO: 41), or LGR6 (e.g., SEQ ID NO: 42).

18. The myeloid cell or mesenchymal cell of any one of aspects 9-17, wherein the transgene is inducible.

19. The myeloid cell or mesenchymal cell of any one of aspects 9-18, wherein the transgene encodes a gene of a inducible gene system, wherein the inducible gene system is a doxorubicin or FK506 binding protein 12 (FKBP) destabilizing domain or protease inducible system.

20. The myeloid cell or mesenchymal cell of aspect 15 or 16, wherein the vector comprises an additional transgene, wherein the additional transgene encodes IL-IRA (e.g., SEQ ID NO: 18), IL-2 (e.g., SEQ ID NO: 19), IL-10 (e.g., SEQ ID NO: 20), IL-12A (e.g., SEQ ID NO: 21), IL-12B (e.g., SEQ ID NO: 22), CXCL9 (e.g., SEQ ID NO: 23), CXCL10 (e.g., SEQ ID NO: 24), SMAD4 (e.g., SEQ ID NO: 25), TGFβ1 (e.g., SEQ ID NO: 26), TGFβ2 (e.g., SEQ ID NO: 27), TGFβ3 (e.g., SEQ ID NO: 28), TREM1 (e.g., SEQ ID NO: 29), sTREM2 (e.g., SEQ ID NO: 5), CD2AP (e.g., SEQ ID NO: 31), FPR2 (e.g., SEQ ID NO: 32), P2RY2 (e.g., SEQ ID NO: 33), P2RY6 (e.g., SEQ ID NO: 34), CHEMR23 (e.g., SEQ ID NO: 35), ERV3-2 (e.g., SEQ ID NO: 36), HERV-W (e.g., SEQ ID NO: 37), HERV-K (e.g., SEQ ID NO: 38), GPR18 (e.g., SEQ ID NO: 39), GPR32 (e.g., SEQ ID NO: 40), GPR37 (e.g., SEQ ID NO: 41), or LGR6 (e.g., SEQ ID NO: 42).

21. The myeloid cell or mesenchymal cell of any one of aspects 6-20, wherein the vector comprises a reporter gene.

22. The myeloid cell or mesenchymal cell of aspect 21, wherein the reporter gene is EGFR, tEGFR, or CD90.1.

23. A method of making a myeloid cell or mesenchymal cell that expresses a protein, the method comprising introducing to the myeloid cell or mesenchymal cell an mRNA encoding IL12, membrane tethered IL12 (mIL12), a IL6 decoy receptor (IL6DR), CD40 Ligand (CD40L), a soluble Triggering Receptor expressed on Myeloid cells 2 decoy receptor (sTREM2), a tissue inhibitor of metalloproteinases (TIMPs), a dominant negative transforming growth factor β receptor II (TGFβRII), a dominant negative transforming growth factor β receptor III (TGFβRIII), or a prostaglandin E2 receptor 2 decoy receptor (EP2 decoy receptor).

24. The method of aspect 23, wherein cDNA is used to create the exogenous mRNA, the cDNA encoding IL12 comprising the sequence of SEQ ID NO: 2, IL6DR comprising the sequence of SEQ ID NO: 3, CD40L comprising the sequence of SEQ ID NO: 4, and sTREM2 comprising the sequence of SEQ ID NO: 6.

25. The method of aspect 23 or 24, wherein the exogenous mRNA comprises a 3′ UTR and cDNA is used to create the 3′ UTR, the cDNA comprising the sequence of any one of SEQ ID NOs: 10-16.

26. The method of any one of aspects 23-25, wherein the introduction of the mRNA is through electroporation, liposomes, or microfluidic squeezing.

27. A myeloid cell or mesenchymal cell comprising exogenous mRNA, wherein the exogenous mRNA encodes viral accessory protein x (Vpx).

28. The myeloid cell or mesenchymal cell of aspect 27, wherein the cell is a myeloid cell and the myeloid cell is differentiated from a hematopoietic stem and progenitor cell (HSPC) which can be CD34+.

29. The myeloid cell or mesenchymal cell of aspect 27, wherein the cell is a myeloid cell and the myeloid cell is a genetically modified bone marrow-derived CXCR4⁺ myeloid cell.

30. The myeloid cell or mesenchymal cell of any one of aspects 27-29, wherein cDNA is used to create the exogenous mRNA encoding Vpx, the cDNA comprising the sequence of SEQ ID NO: 43.

31. The myeloid cell or mesenchymal cell of any one of aspects 27-30, wherein the exogenous mRNA comprises a 3′ UTR and cDNA is used to create the 3′ UTR, the cDNA comprising the sequence of any one of SEQ ID NOs: 10-16.

32. A method of making a myeloid cell that expresses a protein, the method comprising introducing to the myeloid cell an mRNA encoding viral accessory protein x (Vpx).

33. The method of aspect 32, wherein the exogenous mRNA comprises a 3′ UTR and cDNA is used to create the 3′ UTR, the cDNA comprising the sequence of any one of SEQ ID NOs: 10-16.

34. The method of aspect 32 or 33, wherein the introduction of the mRNA is through electroporation, liposomes, or microfluidic squeezing.

35. A method of genetically modifying a myeloid cell or mesenchymal cell, the method comprising
(a) introducing to the cell an mRNA encoding viral accessory protein x (Vpx), and
(b) transducing the cell with a vector.

36. The method of aspect 35, wherein the introduction of the mRNA is through electroporation, liposomes, or microfluidic squeezing.

37. The method of aspect 35 or 36, wherein cDNA is used to create the exogenous mRNA encoding Vpx, the cDNA comprising the sequence of SEQ ID NO: 43.

38. The method of any one of aspects 35-37, wherein the exogenous mRNA comprises a 3′ UTR and cDNA is used to create the 3′ UTR, the cDNA comprising the sequence of any one of SEQ ID NOs: 10-16.

39. A myeloid cell or mesenchymal cell, wherein the cell has been genetically modified to inactivate, knockdown, or remove S100A8 (e.g., SEQ ID NO: 44), S100A9 (e.g., SEQ ID NO: 45), ARG1 (e.g., SEQ ID NO: 46), IDO1 (e.g., SEQ ID NO: 47), IL4 (e.g., SEQ ID NO: 48), TGFβ1 (e.g., SEQ ID NO: 49), TGFβ2 (e.g., SEQ ID NO: 50), TGFβ3 (e.g., SEQ ID NO: 51), ADAM17 (e.g., SEQ ID NO: 52), CD39 (e.g., SEQ ID NO: 53), CD73 (e.g., SEQ ID NO: 54), CD274, CYBB/gp91phox/NOX2 (e.g., SEQ ID NO: 55), BACE1 (e.g., SEQ ID NO: 56), NCKAP1L (e.g., SEQ ID NO: 57), TREM2 (e.g., SEQ ID NO: 30), EP2 (e.g., SEQ ID NO: 58), IL12A (e.g., SEQ ID NO: 21), IL12B (e.g., SEQ ID NO: 22), or TNFA.

40. A method of making a genetically modified myeloid cell or mesenchymal cell, the method comprising using CRISPR to inactivate, knockdown, or remove S100A8 (e.g., SEQ ID NO: 44), S100A9 (e.g., SEQ ID NO: 45), ARG1 (e.g., SEQ ID NO: 46), IDO1 (e.g., SEQ ID NO: 47), IL4 (e.g., SEQ ID NO: 48), TGFβ1 (e.g., SEQ ID NO: 49), TGFβ2 (e.g., SEQ ID NO: 50), TGFβ3 (e.g., SEQ ID NO: 51), ADAM17 (e.g., SEQ ID NO: 52), CD39 (e.g., SEQ ID NO: 53), CD73 (e.g., SEQ ID NO: 54), CD274, CYBB/gp91phox/NOX2 (e.g., SEQ ID NO: 55), BACE1 (e.g., SEQ ID NO: 56), NCKAP1L (e.g., SEQ ID NO: 57), TREM2 (e.g., SEQ ID NO: 30), EP2 (e.g., SEQ ID NO: 58), IL12A (e.g., SEQ ID NO: 21), IL12B (e.g., SEQ ID NO: 22), or TNFA.

41. A method of treating a mammal having a tumor, the method comprising administering to the mammal a therapeutically effective amount of (a) a myeloid cell or mesenchymal cell of any one of aspects 1-22, 27-31, and 39 or (b) a myeloid cell or mesenchymal cell produced by the method of any one of aspects 23-26, 32-38, and 40.

42. The method of aspect 41, wherein the myeloid cell or mesenchymal cell is cryopreserved and thawed before administration.

43. The method of aspect 42 or 43, wherein the tumor is a brain tumor, a pancreatic adenocarcinoma, or an osteosarcoma.

44. A method of treating a mammal having a neurodegenerative condition, an autoimmune disorder, or an inflammatory disorder, the method comprising administering to the mammal a therapeutically effective amount of (a) a myeloid cell or mesenchymal cell of any one of aspects 1-22, 27-31, and 39 or (b) a myeloid cell or mesenchymal cell produced by the method of any one of aspects 23-26, 32-38, and 40.

45. The method of aspect 44, wherein the myeloid cell or mesenchymal cell is cryopreserved and thawed before administration.

46. A method of treating a mammal having a cancer, the method comprising administering to the mammal a therapeutically effective amount of (a) a myeloid cell or mesenchymal cell of any one of aspects 1-22, 27-31, and 39 or (b) a myeloid cell or mesenchymal cell produced by the method of any one of aspects 23-26, 32-38, and 40.

47. The method of aspect 46, wherein the myeloid cell or mesenchymal cell is cryopreserved and thawed before administration.

48. The method of aspect 46 or 47, wherein the method further comprises administering an additional therapeutic treatment, wherein the additional therapeutic treatment comprising chemotherapy, surgical resection of a tumor, treatment with targeted cancer therapy, allogeneic or autologous stem cell transplantation, T cell adoptive transfer, chimeric antigen receptor (CAR)-modified T cells, T cell receptor (TCR)-modified T cells, a dendritic cell vaccine, an oncolytic virus, a small molecule, a monoclonal antibody or antigen binding fragments thereof, hormone-blocking therapy, radiation therapy, other immunotherapies, or myeloid-targeting pre-conditioning regimens optionally comprising clodronate or CSF1R inhibitors.

49. The method of aspect 48, wherein the additional therapeutic treatment is chimeric antigen receptor (CAR)-modified T cells.

50. A nucleotide sequence for a 3′ UTR that improves the stability of a mRNA sequence, wherein cDNA is used to create the 3′ UTR sequence, and the cDNA comprises the sequence of KJ1 (SEQ ID NO: 6), mtRNR1-KJ1 (SEQ ID NO: 7), mtRNR1-AES-KJ1 (SEQ ID NO: 8), KJ2 (SEQ ID NO: 9), mtRNR1-KJ2 (SEQ ID NO: 10), mtRNR1-AES-KJ2 (SEQ ID NO: 11), or KJ3 (SEQ ID NO: 12).

It shall be noted that the preceding are merely examples of aspects of the disclosure. Other exemplary aspects are apparent from the entirety of the description herein. It will also be understood by one of ordinary skill in the art that each of these aspects may be used in various combinations with the other aspects provided herein.

The following examples further illustrate the disclosure but, of course, should not be construed as in any way limiting its scope.

### Example 1

This example demonstrates expression of mRNA-encoded cargo in human genetically engineered myeloid cells (GEMys).

In solid tumors, immunotherapeutic strategies have been limited by the ability of T cells to penetrate deep into tumors and to persist, due to suppression by myeloid cells accumulating in tumor microenvironment (TME). Myeloid cells are part of the innate immune system and are multifunctional. They can be directly cytotoxic, capable of antigen presentation and efficient at tissue repair. During repair, these cells dampen immune response to prevent immune-mediated tissue damage. These cells are the first responders to damaged tissue sites and the most abundant cell in the tumor microenvironment.

Primary human monocytes from healthy donors were electroporated without mRNA (blank), GFP mRNA, human single chain IL12 mRNA, and tEGFR-IL12 mRNA (wherein the cDNA used to create the mRNA comprises SEQ ID NO: 64), using the Maxcyte electroporation device. Electroporated cells were treated with protein transport inhibitors (brefeldin A and monensin) immediately after electroporation for 14 hours. Cells were fixed, permeabilized, and intracellular staining for IL-12 was performed. Data was analyzed for EGFR, GFP, and IL12 expression by flow cytometry.

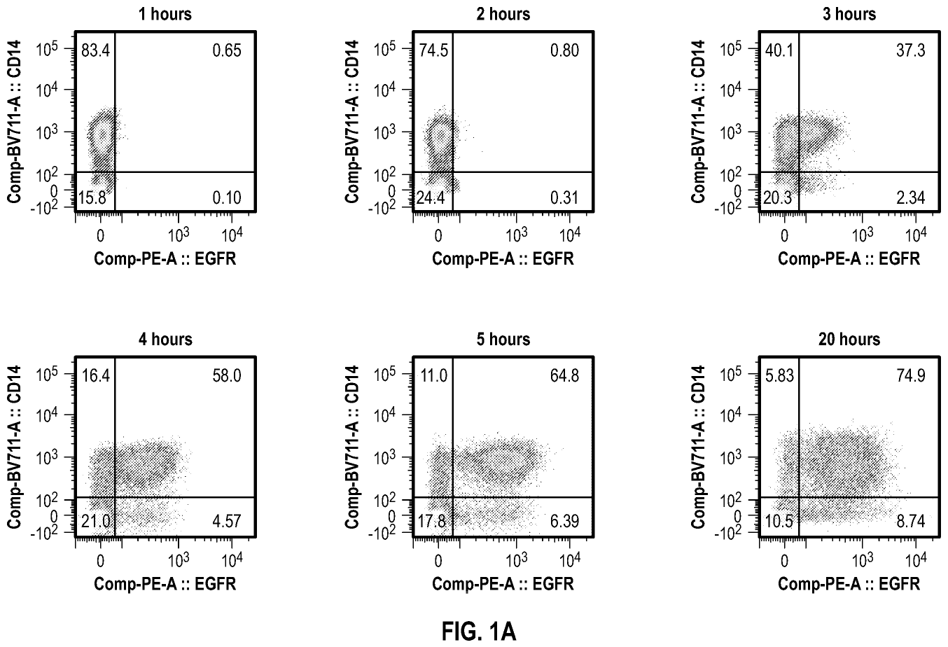

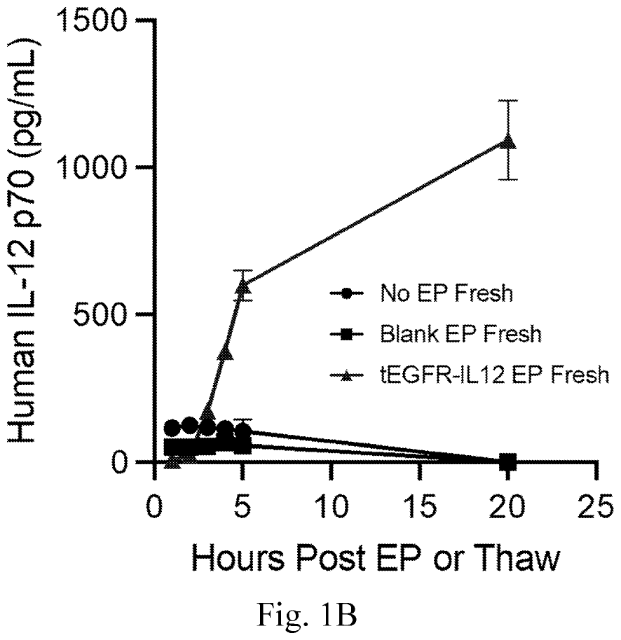

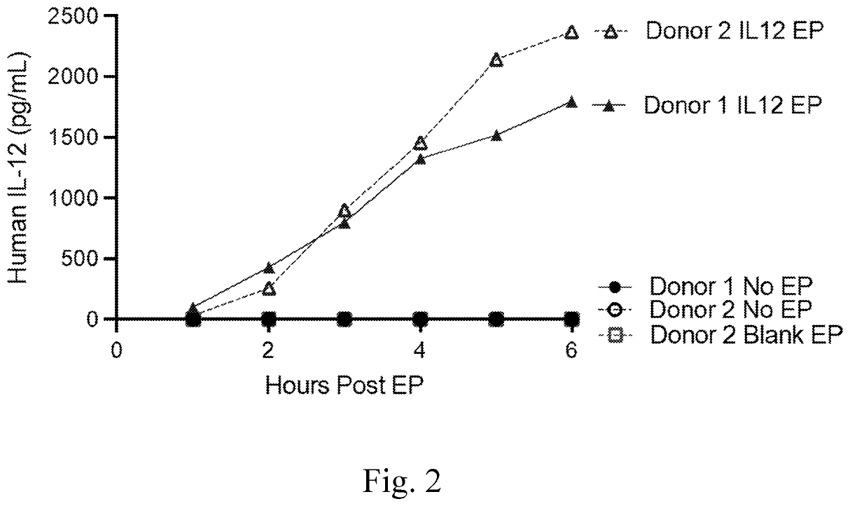

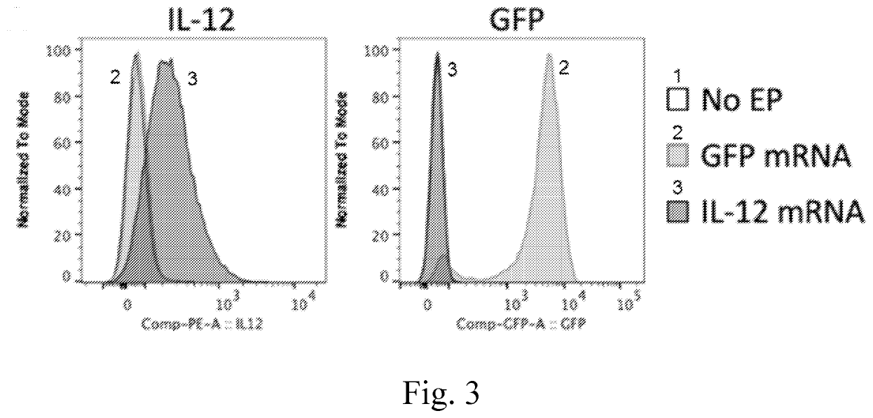

Flow cytometry analysis showed that EGFR mRNA electroporation increased the expression levels of EGFR mRNA in primary human monocytes over the following 20 hours (FIG. 1A). In comparison primary human monocytes that underwent no electroporation treatment or electroporation treatment without mRNA (blank) did not show a comparable increase in EGFR expression (FIG. 1B). Similar results were observed with IL12 expression levels which increased in primary human monocytes that were electroporated with IL12 mRNA, while those cells that were not electroporated or electroporated without mRNA (blank) or GFP mRNA show no increase in IL12 expression levels (FIG. 2 and FIG. 3). Primary human monocytes that underwent electroporation with GFP mRNA showed increased GFP expression relative to those cells that were not electroporated or electroporated with IL12 mRNA (FIG. 3).

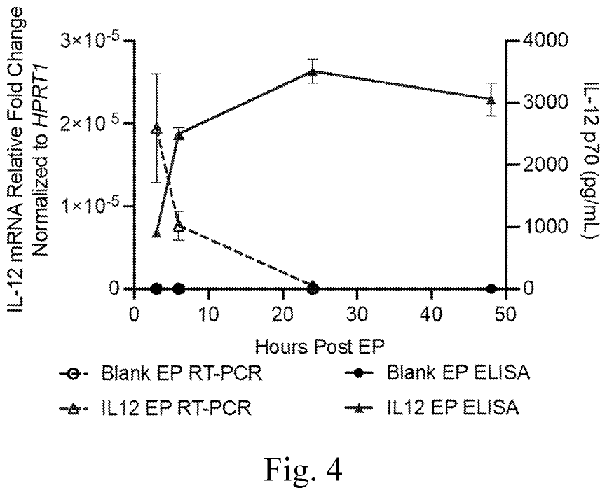

RT-PCR analysis measuring IL12 expression levels normalized to hypoxanthine-guanine phosphoribosyltransferase (HPRT1) showed increased expression in primary human monocytes electroporated with IL12 mRNA relative to those electroporated without mRNA (blank) over the following 24 hours (FIG. 4). IL12 mRNA levels appeared to peak around 5 hours after electroporation and continued to decrease until none were detected at approximately 24 hours after electroporation. ELISA analysis showed similar results with increased IL12 protein levels in primary human monocytes electroporated with IL12 mRNA relative to those electroporated with blank mRNA (blank) over the following 48 hours (FIG. 4). IL12 protein levels appeared to peak around 24 hours after electroporation and had decreased slightly by 48 hours after electroporation.

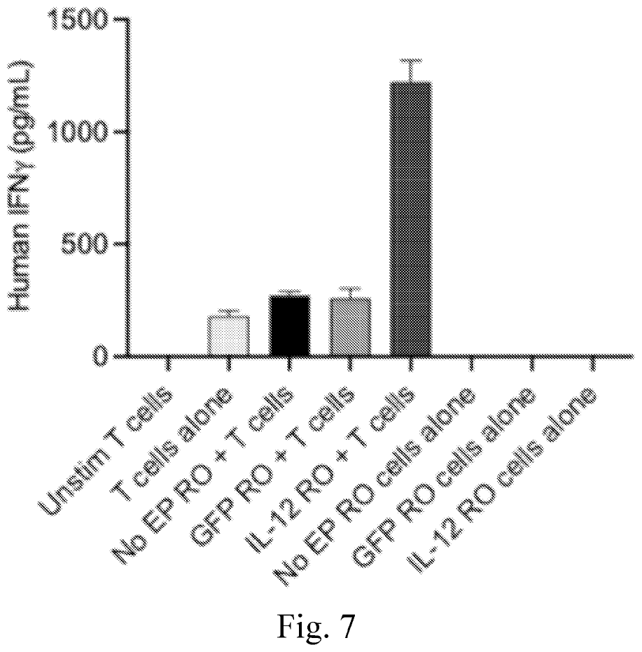

To test the ability of IL12-mRNA-GEMys to induce lymphocyte activation, mRNA electroporated monocytes (RO elutriation fraction) were co-cultured with donor-matched T cell lymphocytes. IFNγ production was measured by ELISA analysis at 24 hours in unstimulated T cells (Unstim), T cells alone, No EP RO+T cells, GFP RO+T cells, IL12 RO+T cells, No EP RO cells alone, GFP RO cells alone, and IL12 RO cells alone (FIG. 7). The unstimulated T cells, No EP RO cells alone, GFP RO cells alone, and IL12 RO cells alone showed no IFNγ protein, while T cells alone, No EP RO+T cells, and GFP RO+T cells showed low levels of IFNγ protein (approximately 200 μg/mL), while IL12 RO+T cells showed a high level of IFNγ protein (approximately 1300 μg/mL). Human myeloid cells engineered to express IL12 induced the most IFNg production by co-cultured T cells lymphocytes.

These results suggest that primary human monocytes electroporated with mRNA constructs will increase production of the protein encoded by the mRNA. These results also suggest that the protein is trafficked and functions normally in the cell. This suggests that GEMys can be successfully produced by such a method.

### Example 2

This example demonstrates introduction of Viral protein x (Vpx) mRNA into human monocytes, which improves lentiviral expression.

Stable expression of a gene product across an expanding population can require lentiviral transduction. However, transduction of myeloid cells with viral components for stable insertion of DNA into the cell's genome has proven challenging due to the increased presence of antiviral mechanisms in myeloid cells. Although myeloid cells can be transfected by, e.g., adenoviral vectors, the introduction of the adenoviral vector is a strong activator of the immune response and can skew the myeloid cell to an inflammatory phenotype as a result of an interferon response. Such activation of an immune response may not be desirable in certain disease settings and may lead to immune mediated removal of these engineered cells.

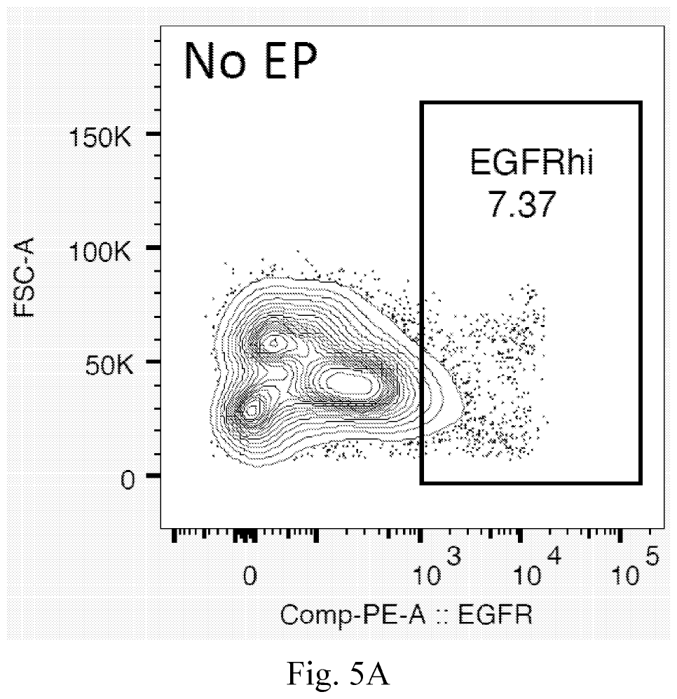

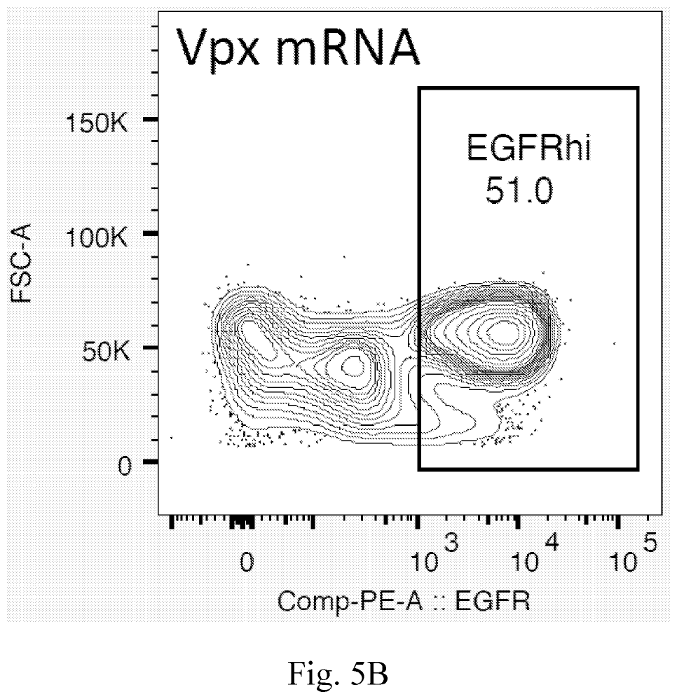

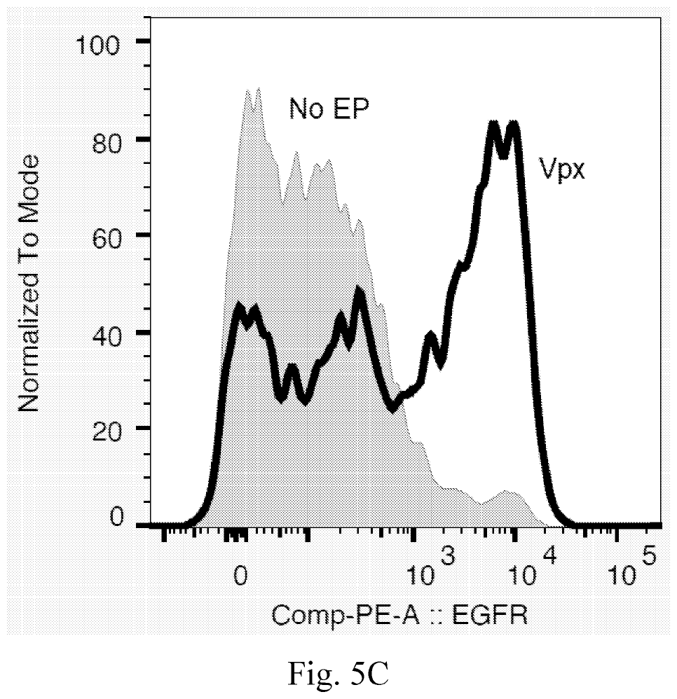

Primary human monocytes were not electroporated or electroporated with mRNA encoding the antiviral protein Vpx. After 24 hours, cells were transduced with lentivirus encoding human truncated EGFR (tEGFR) and IL-12. EGFR^{high} expression on the surface is shown as a readout of transduction efficiency 24 hours post-transduction (FIGS. 5A-5B). The primary human monocytes that were electroporated with Vpx mRNA showed increased expression of EGFR relative to those that were not electroporated (FIG. 5C). There is minimal lentiviral transduction of EGFR IL12 vector with only 7% expression of EGFR when transduced without Vpx. Without wishing to be bound by theory, it is believed that Vpx expression inhibits SAMHD1, a protein responsible for preventing efficient lentiviral integration into myeloid cells.

These results suggest that expression of Vpx in potential GEMys could increase expression of the cargo proteins introduced by subsequent lentiviral transduction.

### Example 3

This example demonstrates that mRNA-GEMys may be cryopreserved and still express mRNA after thawing, both in vitro and in vivo.

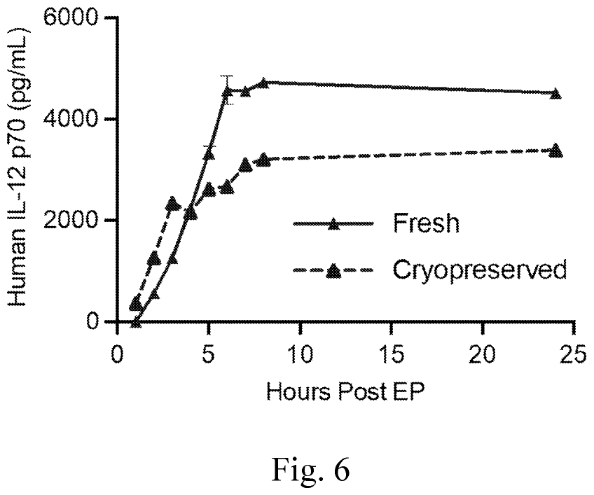

Human GEMys were prepared as in example 1. The human GEMys were then cryopreserved by resuspending the cell in Plasmalyte immediately following mRNA electroporation and adding one volume of CryoStor CS10 freezing media. Cells were frozen slowly at −80 C in a isopropanol bath and transferred to liquid nitrogen for extended storage. Cryopreserved GEMys were thawed in a 37C water bath followed by the addition of 1 mL pre-warmed culture media incubated for one minute, addition of 2 mL pre-warmed culture media for one minute, addition of 4 mL culture media for one minute, and then spun down and resuspended in culture media. ELISA analysis was then performed on fresh and thawed GEMys over the 24 hours following electroporation or thawing (FIG. 6). Both the fresh and thawed cryopreserved GEMys showed increasing IL12 protein levels for approximately 10 hours and showed comparable levels at 24 hours.

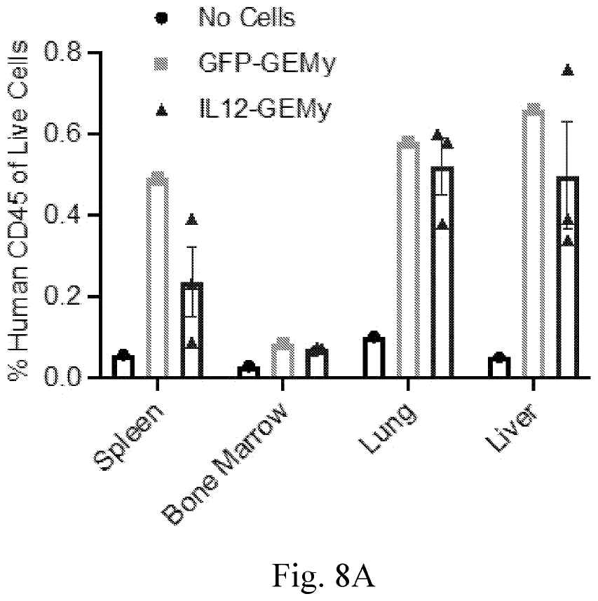

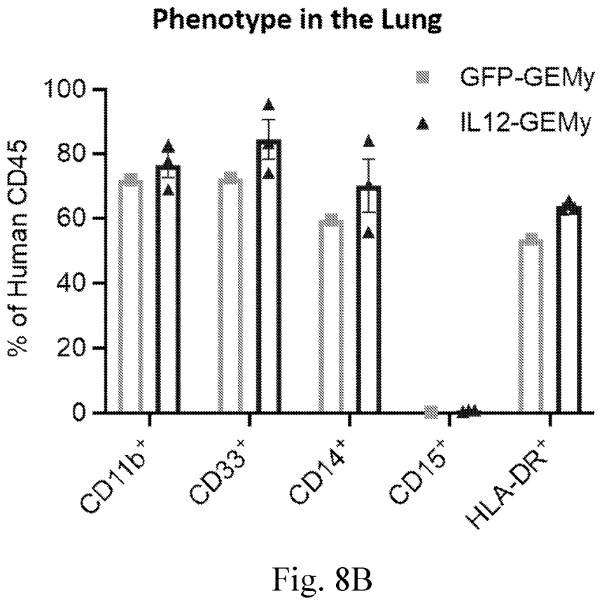

Fresh GFP-GEMys and IL12-GEMys were injected intravenously into osteosarcoma tumor-bearing NSG-SGM3 mice. The next day after GEMys injection, mice were sacrificed and spleen, bone marrow, lung and liver samples were extracted. These samples were digested and mechanically processed into single cell suspension and staining for human CD45 was performed and analyzed by flow cytometry. The lung tissue was also stained for human CD11b, CD33, CD15, and HLA-DR. Analysis of the tissue samples showed increased localization of the GEMys to the lung, liver, and spleen compared to the bone marrow (FIG. 8A). Phenotypic analysis showed that over 50% of the GEMys in the lung were CD11b, CD33, CD14 and HLDA-DR positive cells, while almost none were CD15 positive (FIG. 8B). The data demonstrate the tissue localization and the myeloid (CD11b+, CD33+, HLA-DR+) and classical monocyte (CD14+) cell phenotype of the human mRNA-engineered GEMys after administration in vivo in a mouse model.

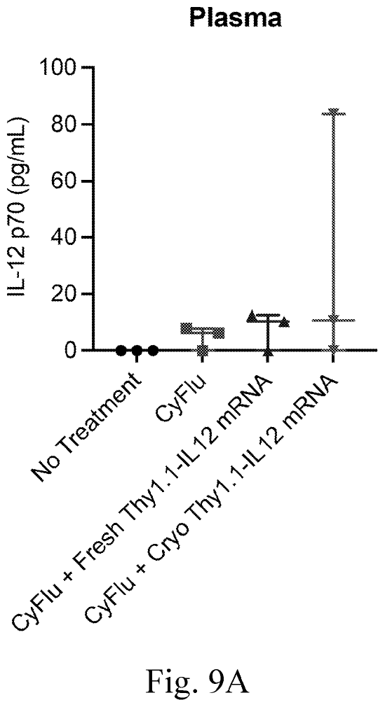

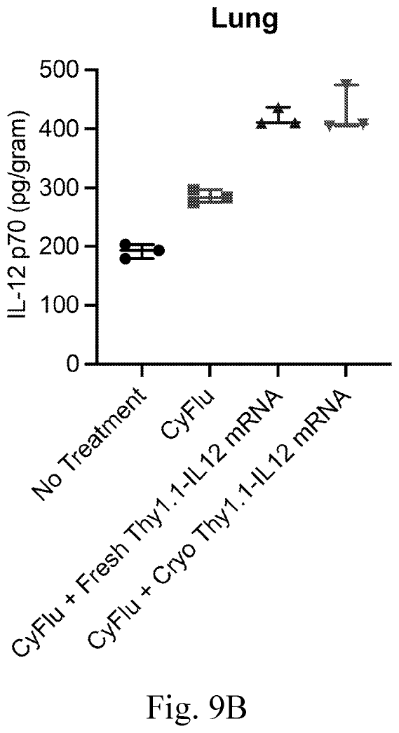

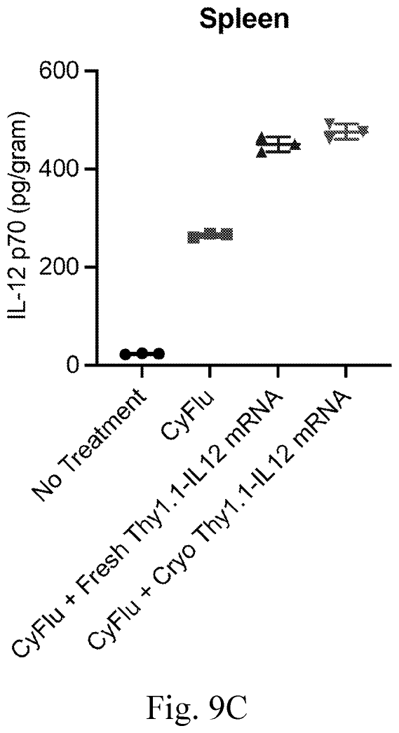

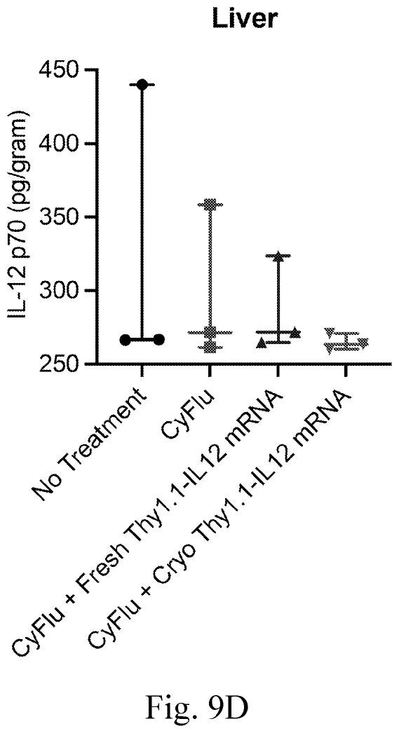

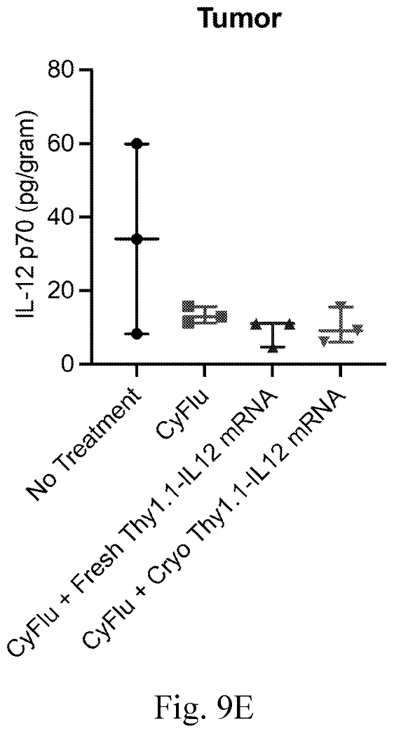

Syngeneic M3-9-M Rhabdomyosarcoma tumor bearing C57/B16 mice were given no treatment, or treated by injection with Cy/Flu alone, Cy/Flu and freshly prepared Thy1.1 IL12 mRNA murine GEMys, or Cy/Flu and thawed cryopreserved Thy1.1 IL12 mRNA murine GEMys. The day after injection the mice were sacrificed and spleen, bone marrow, lung, liver, and tumor samples were extracted. These samples were flash frozen and analysed by ELISA for IL12 protein levels. The plasma samples with no treatment contained no detectable IL12, while the Cy/Flu, Cy/Flu and fresh GEMys, and Cy/Flu and cryopreserved GEMys had low but detectable amounts of IL12 protein, about 10 μg/mL, with one mouse in the cryopreserved GEMys group had around 80 μg/mL IL12 detected in the plasma (FIG. 9A). The lung samples from the Cy/Flu and fresh GEMys treatment and Cy/Flu and cryopreserved GEMys treatment had increased IL12 protein levels, about 400 pg/gram, compared to the treatment with Cy/Flu alone, about 300 pg/gram, or no treatment, about 200 pg/gram (FIG. 9B). Similarly, the spleen samples from the Cy/Flu and fresh GEMys treatment and Cy/Flu and cryopreserved GEMys treatment had increased IL12 protein levels, about 500 pg/gram, compared to the treatment with Cy/Flu alone, about 250 pg/gram, or no treatment, which was undetectable (FIG. 9C). The liver samples showed highly variable but similar levels of IL. 12 protein, about 260 pg/gram median among all treatments (FIG. 9D). The tumor samples showed higher levels of IL12 protein in the no treatment group, about 35 mg/mL, compared to the other treatment groups, which all had less than half as much IL12 protein (FIG. 9E). Elevated IL12 levels in the mice treated with IL12-mRNA-GEMys demonstrate the production of the IL12 from the mRNA in vivo after injection of these engineered cells into mice. The similar levels of IL12 protein levels among these tissue samples for the fresh and cryopreserved GEMys treatments suggest that cryopreserved GEMys function comparably to fresh GEMys in vivo which allows for administration of a cryopreserved GEMy product to allow for greater flexibility in timing and prevent cell loss that can occur with prolonged cell culture.

These results suggest that mRNA-GEMys may be cryopreserved and still maintain the ability to express mRNA after thawing in both in vitro and in vivo systems. This suggests that mRNA-GEMys could successfully be stored long term and thus be ready for use shortly after thawing.

### Example 4

This example demonstrates the preparation of CD40L-GEMys and use thereof for induction of functional CD40 signaling.

The early metastatic microenvironment is T cell poor. CD4⁺ T cells are decreased in this pro-tumorigenic environment. CD40L is found on the surface of T cells or soluble when cleaved from the immune cell surface, and it activates CD40 expressed on B cells and dendritic cells (DCs) to stimulate helper T cells and other immune cells. CD40L is expressed on helper T cells that do not traffic well to the interior TME.

CD40L-GEMys were developed to signal to CD40-expressing B cells and DCs to activate and aid in recruitment and activation of T cells to promote antitumor immunity. The cDNA used to create the murine CD40L mRNA comprises the sequence of SEQ ID NO: 68. In addition, CD40L can activate B cells by providing a helper T cell signal for germinal center formation, isotype class switching and production of immunoglobulin antibodies.

Murine bone marrow derived cells were either untransduced or transduced with Thy 1.1 mRNA or CD40 ligand (CD40L) lentivirus and differentiated into the GEMys product over 4 days in culture with SCF, IL-6, and FLT3L. These GEMys were cultured with either the CD40L reporter line, HEK-Blue™ CD40L, or splenocytes in a 1:1 ratio. Cells were then collected for analysis.

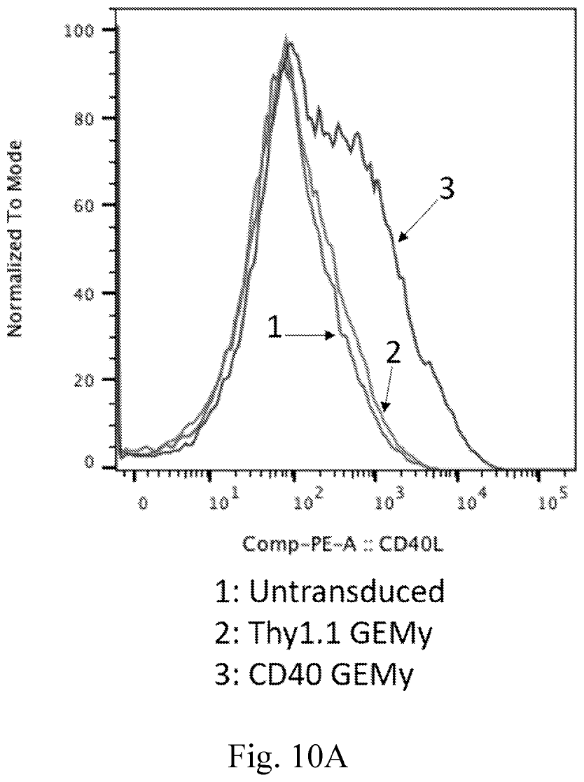

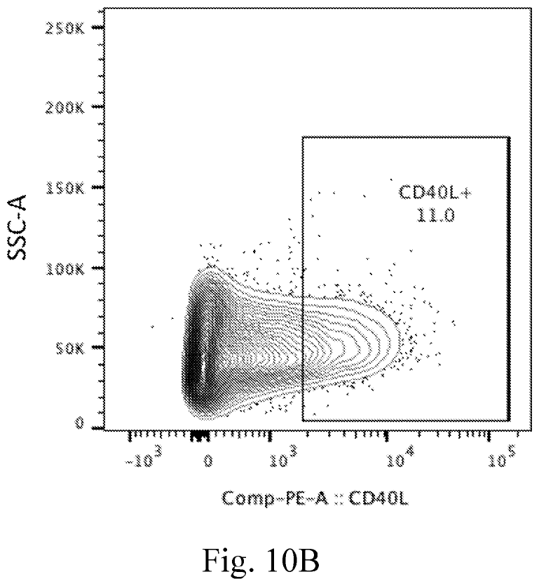

Flow cytometry analysis of CD40L protein levels showed that CD40L GEMys had higher levels of CD40L than either the untransduced or Thy1.1 GEMys samples (FIG. 10A). FIG. 10B shows the expression of CD40L mRNA in the CD40L GEMys.

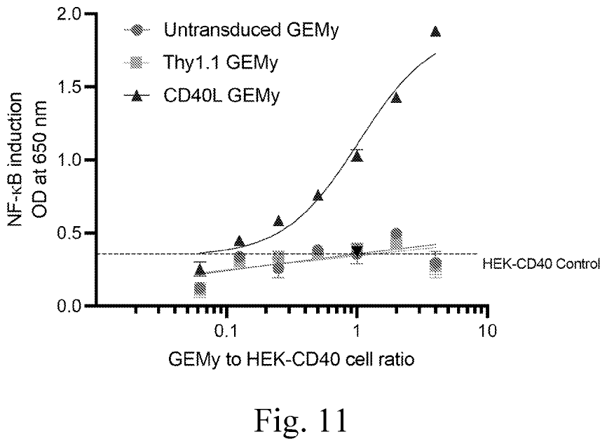

To determine if the CD40L GEMys induced activation of CD40 signaling the induction of nuclear factor kappa-light-chain-enhancer of activated B cells (NF-κB) was measured in the HEK-Blue™ CD40L reporter cell line, which produce SEAP in response to CD40 signaling. QUANTI-Blue™ solution was then used to detect SEAP levels according to manufacturers instructions in the treated HEK-Blue™ CD40L cells and HEK-CD40 cells as a negative control. The HEK-Blue™ CD40L cells cultured with CD40L GEMys showed increased CD40 signaling compared to cells treated with the untransduced and Thy1.1 GEMys which showed similar levels to the HEK-CD40 control (FIG. 11). This suggests that CD40L GEMys are able to induce functional CD40 signaling.

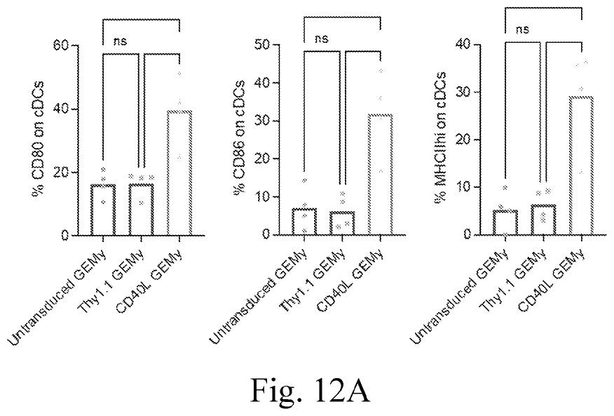

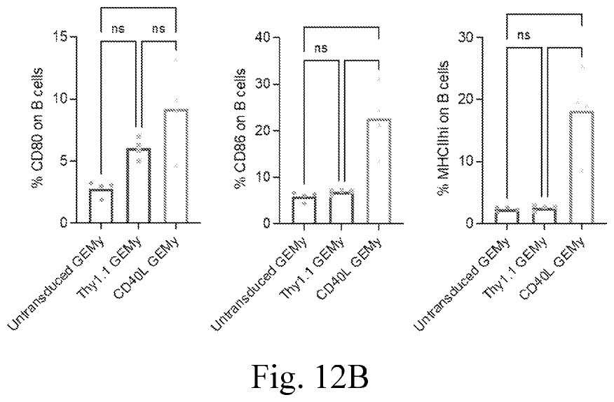

To determine if CD40L GEMys can activate B cells and dendritic cells, splenocytes were co-cultured with either untransduced, Thy1.1, or CD40L GEMys and then staining for CD80, CD86, and MHCII was performed and analyzed by flow cytometry. The splenocytes treated with CD40L GEMys showed a significantly higher percent of CD80, CD86, and MHCII positive cells among the classical dendritic cells than those treated with untransduced or Thy1.1 GEMys (FIG. 12A). Similarly, splenocytes treated with CD40L GEMys showed a significantly higher percent of CD86 and MHCII positive cells among the B cells than those treated with untransduced or Thy1.1 GEMys, and while the trend held for CD80 positive cells as well the difference was only significant for untransduced GEMys (FIG. 12B). This suggests that CD40L GEMys are able to induce functional CD40 signaling and activate DCs and B cells.

These results suggest that CD40L GEMys could be an effective therapeutic to promote antitumor immunity.

### Example 5

This example demonstrates the preparation of sTREM2-GEMys and use thereof for antitumor immunity.

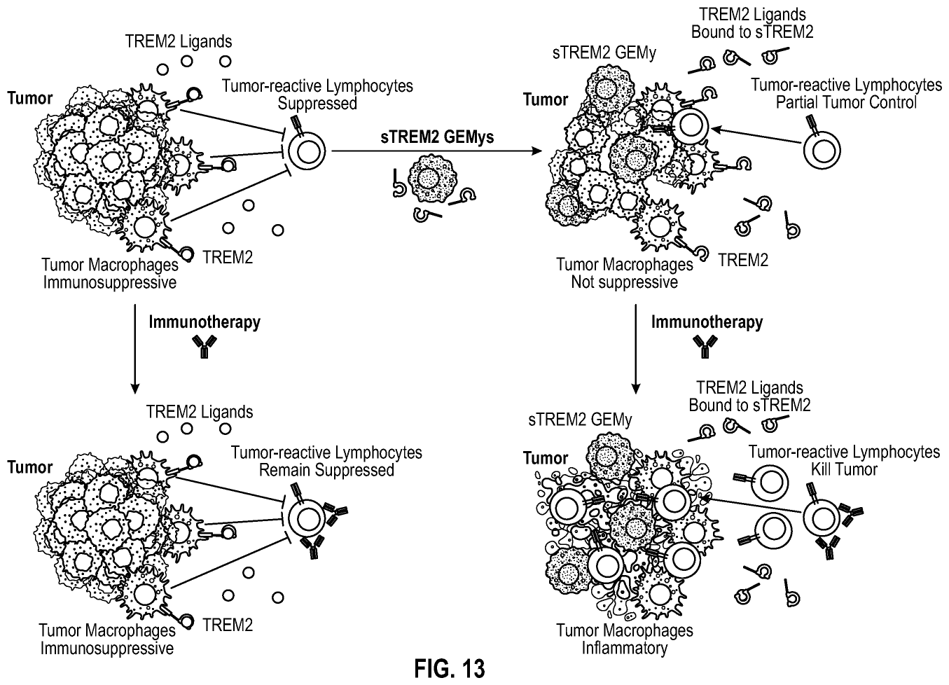

In the tumor microenvironment TREM2 intracellular signaling induces immune suppression and can contribute to tumor metastasis. A soluble TREM2 receptor decoy (sTREM2) could inhibit TREM2 signalling and limit tumor progression. This proposed mechanism is shown in FIG. 13.

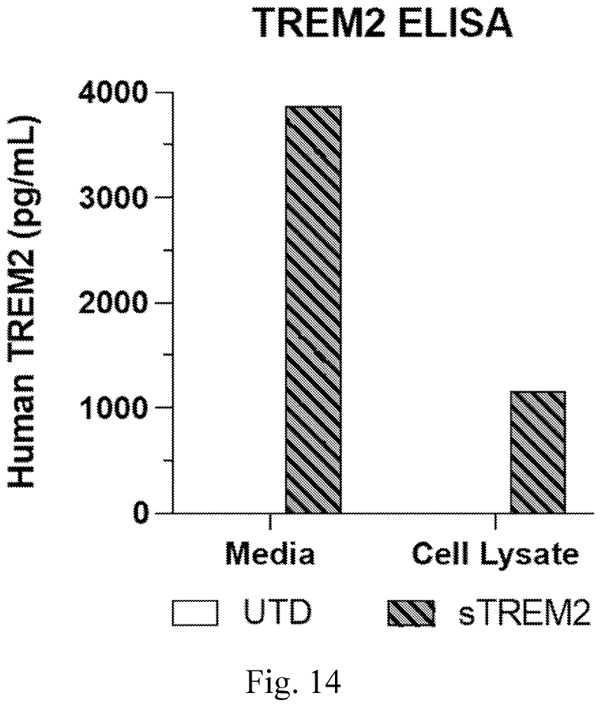

GEMys cells were prepared as in Example 2, but using murine cells and human STREM2 mRNA. ELISA analysis for TREM2 was then performed on the media and cell lysate of untransduced (UTD) and sTREM2 transduced GEMys. The untransduced media and lysate showed no detectable levels of TREM2 protein while the sTREM2 GEMys showed detectable levels of TREM2 in both the media and cell lysate (FIG. 14).

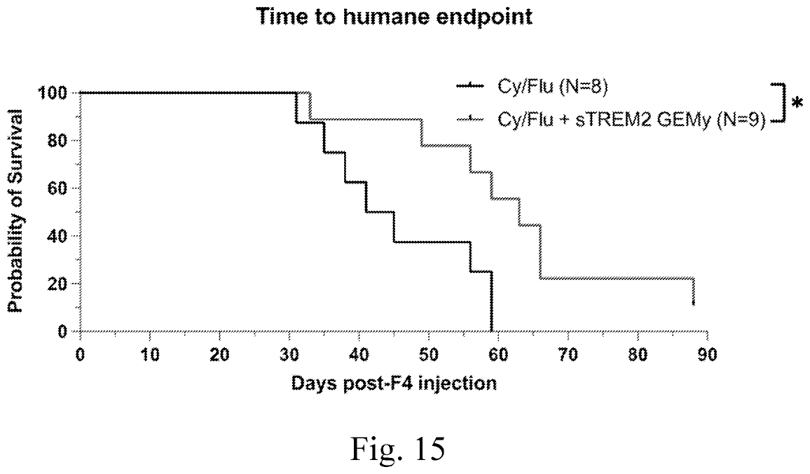

To test the efficacy of sTREM2 GEMys for antitumor immunity, F4 osteosarcoma tumor bearing C57BL/6 mice were injected with Cy/Flu alone or in combination with sTREM2 GEMys and then probability of survival was measured to a humane endpoint (FIG. 15). sTREM2 GEMys injected mice had significantly increased probability of survival, living an average 88 days post F4 osteosarcoma cell injection as opposed to the average 59 days for mice not injected with sTREM2 GEMys.

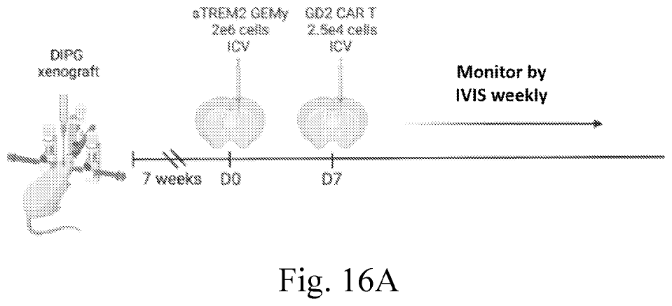

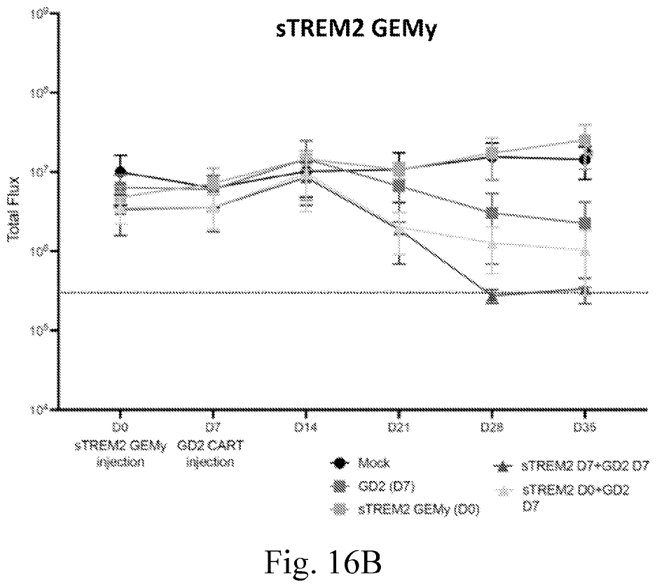

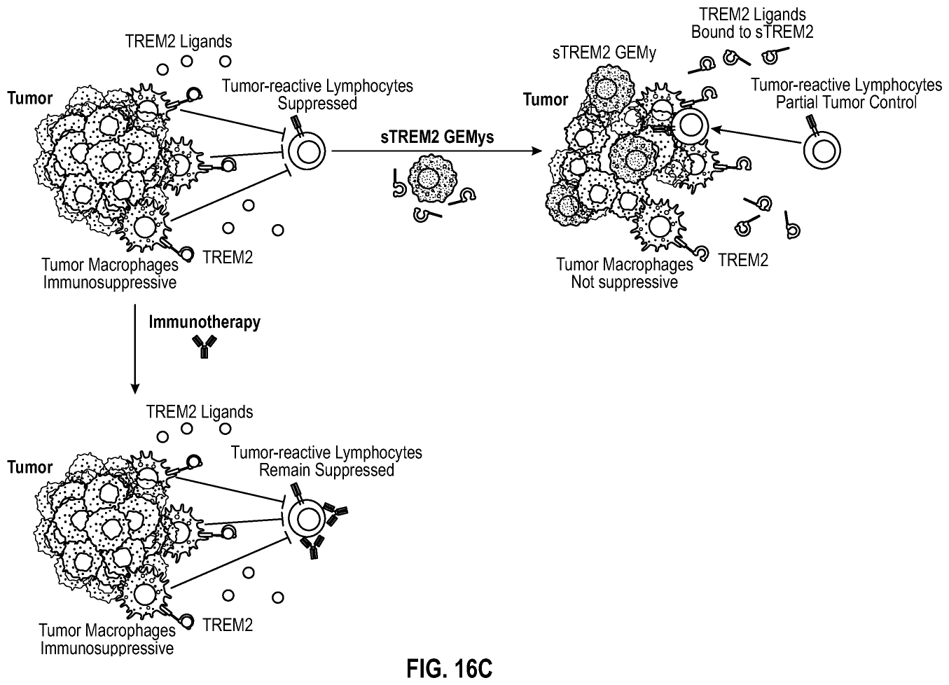

To test the efficacy of sTREM2 GEMys in combination with chimeric antigen receptor T cells for antitumor immunity, NSG mice bearing midline glioma also known as diffuse intrinsic pontine glioma (DIPG) were treated as shown in the experimental design of FIG. 16A. These DIPG bearing NSG mice were given either a mock treatment, or treated by a intracerebroventricular administration of: sTREM2 GEMy alone at Day 0, GD2 CART alone at Day 7, a combination of sTREM2 GEMys with subtherapeutic dosing of GD2 CART given together at Day 7, or a combination of sTREM2 GEMys given at Day 0 and GD2 CART given at Day 7 (FIG. 16B). The treatments consisting of a combination of sTREM2 GEMy and GD2 CART showed the most effective antitumor immunity. The sTREM2 GEMy and GD2 CART combination administered at the same time, Day 7, even showed no more tumor measurable (dashed line) at Day 28. Without wishing to be bound by theory, the proposed mechanism by which sTREM2 in combination with immunotherapy alleviates myeloid mediated immune suppression to improve anti-tumor T cell efficacy, which allows for either decreased dosing or improved efficacy with standard dosing is shown in FIG. 16C.

These results suggest that sTREM2 GEMys could be an effective therapeutic alone or in combination with other immunotherapies to promote antitumor immunity.

### Example 6

This example demonstrates myeloid specific promoters limit expression of cargo to the myeloid cell compartment.

Myeloid specific synthetic promoters have been developed to enhance cargo expression in myeloid cells by lentiviral vectors, either in differentiated monocytes or for transduction of CD34+ hematopoietic stem and progenitor cells. This is to ensure expression of a cargo protein is restricted to myeloid populations and not expressed significantly in immature hematopoietic populations.

Several truncated promoters from myeloid specific transcription factors have been developed to use for effective expression of a downstream sequence to result in protein expression in myeloid cells. Myeloid synthetic promoters have been created with truncated components of myeloid promoters such as Pu1 and C/EBPa.

A myeloid specific synthetic promoter has been developed based on highest expression in bone marrow-derived myeloid cells and primary human monocytes that home to tissue specific sites and drive expression of a protein of interest. The expression of desired cargo proteins can be restricted to myeloid cells with improved expression in myeloid cells by using the myeloid specific synthetic promoter, which includes truncated myeloid specific transcription factor promoters SP1, Pu.1a, Pu.1b, C/EBPa, AP1, AML-1, and a mutated MMP14 promoter.

These results suggest that these promoters could be used to drive expression specific to myeloid cells and therefore be used to more effectively target tumors and promote antitumor immunity.

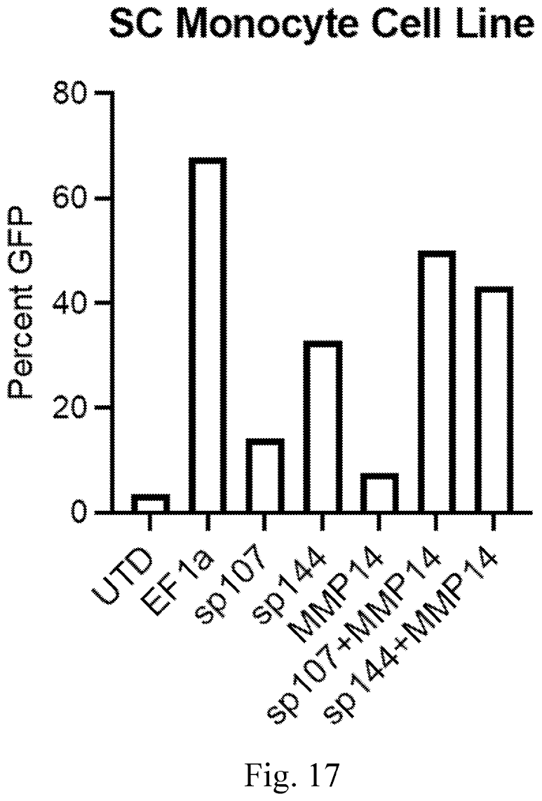

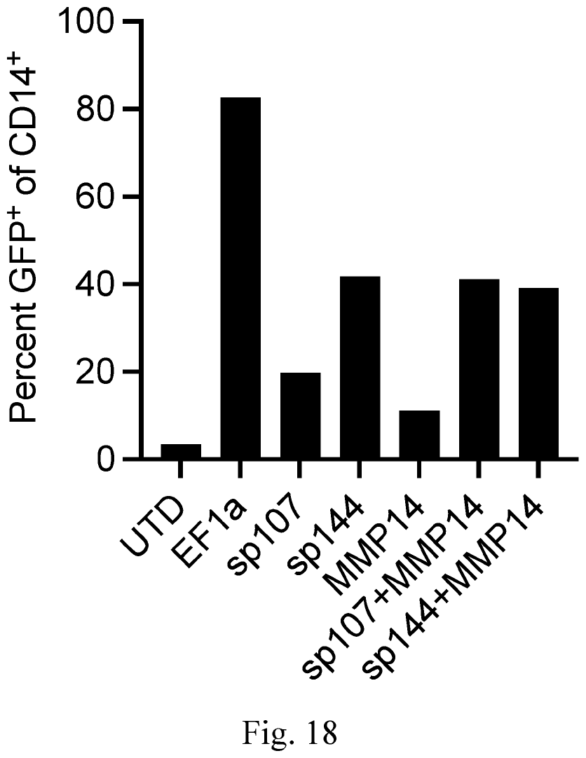

SC monocyte cell lines and primary human monocytes cultures were either untransduced or underwent treatment with a lentiviral vector with either a general promoter EF1a or myeloid specific promoters sp107, sp144, MMP14, combined sp107 and MMP14, or combined sp144 and MMP14 promoter sequences driving expression of a GFP reporter. The percent of GFP positive cells were analysed for each group In the SC monocyte cell lines both the sp107+MMP14 and sp144+MMP14 promoters showed increased expression over the myeloid specific promoters alone (FIG. 17). In the primary human monocyte culture the percent of GFP positive CD14 positive cells were comparable for the sp 144, combined sp107 and MMP14, or combined sp144 and MMP14 promoter sequences, about 40%, which was higher than either sp107 or MMP14 alone (FIG. 18).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A myeloid cell or mesenchymal cell comprising exogenous mRNA, wherein the exogenous mRNA encodes IL12, membrane tethered IL12 (mIL12), a IL6 decoy receptor (IL6DR), CD40 Ligand (CD40L), a soluble Triggering Receptor expressed on Myeloid cells 2 decoy receptor (sTREM2), a tissue inhibitor of metalloproteinases (TIMPs), a dominant negative transforming growth factor β receptor II (TGFβRII), a dominant negative transforming growth factor β receptor III (TGFβRIII), or a prostaglandin E2 receptor 2 decoy receptor (EP2DR).

2. The myeloid cell or mesenchymal cell of claim 1, wherein the cell is a myeloid cell and the myeloid cell is differentiated from a hematopoietic stem and progenitor cell (HSPC).

3. The myeloid cell or mesenchymal cell of claim 1, wherein the cell is a myeloid cell and the myeloid cell is a genetically modified CD34+ bone marrow-derived CXCR4+ myeloid cell.

4. The myeloid cell or mesenchymal cell of claim 1, wherein cDNA is used to create the exogenous mRNA, the cDNA encoding IL12 comprising the sequence of SEQ ID NO: 2, IL6DR comprising the sequence of SEQ ID NO: 3, CD40L comprising the sequence of SEQ ID NO: 4, and sTREM2 comprising the sequence of SEQ ID NO: 5.

5. The myeloid cell or mesenchymal cell of claim 1, wherein the exogenous mRNA comprises a 3′ UTR and cDNA is used to create the 3′ UTR, the cDNA comprising the sequence of any one of SEQ ID NOs: 10-16.

6. The myeloid cell or mesenchymal cell of claim 1, wherein the myeloid cell or mesenchymal cell comprises a vector.

7. The myeloid cell or mesenchymal cell of claim 6, wherein the vector is a plasmid vector, a yeast vector, or a viral vector.

8. The myeloid cell or mesenchymal cell of claim 6, wherein the vector is a lentiviral vector.

9. The myeloid cell or mesenchymal cell of claim 6, wherein the vector comprises a transgene.

10. The myeloid cell or mesenchymal cell of claim 9, wherein the vector comprises an expression control sequence operatively linked to the transgene.

11. The myeloid cell or mesenchymal cell of claim 10, wherein the expression control sequence is a promoter.

12. The myeloid cell or mesenchymal cell of claim 11, wherein the promoter is a hVMD2 promoter, SV40 early promoter, RSV promoter, adenovirus major late promoter, human CMV immediate early I promoter, poxvirus promoter, 30K promoter, I3 promoter, sE/L promoter, 7.5K promoter, 40K promoter, C1 promoter, EF-1a promoter, sp107 promoter, sp144 promoter, MMP14 promoter, sp107 and MMP14 combination promoter, or sp114 and MMP14 combination promoter.

13. The myeloid cell or mesenchymal cell of claim 12, wherein the sp107 promoter comprises SEQ ID NO: 13, the sp144 promoter comprises SEQ ID NO: 14, the MMP14 promoter comprises SEQ ID NO: 15, the sp107 and MMP14 combination promoter comprises SEQ ID NO: 16, and the sp114 and MMP14 combination promoter comprises SEQ ID NO: 17.

14. The myeloid cell or mesenchymal cell of claim 9, wherein the transgene encodes a cytokine, a chemokine, an enzyme, a substrate, a receptor decoy, an antibody, or a gene of a suicide gene system.

15. The myeloid cell or mesenchymal cell of claim 14, wherein the transgene encodes an enzyme, wherein the enzyme is hyaluronidase.

16. The myeloid cell or mesenchymal cell of claim 14, wherein the transgene encodes a gene of a suicide gene system, wherein the suicide gene system is a Herpes Simplex Virus Thymidine Kinase (HSVTK)/Ganciclovir (GCV) suicide gene system or an inducible Caspase suicide gene system.

17. The myeloid cell or mesenchymal cell of claim 9, wherein the transgene encodes IL-IRA (SEQ ID NO: 18), IL-2 (SEQ ID NO: 19), IL-10 (SEQ ID NO: 20), IL-12A (SEQ ID NO: 21), IL-12B (SEQ ID NO: 22), CXCL9 (SEQ ID NO: 23), CXCL10 (SEQ ID NO: 24), SMAD4 (SEQ ID NO: 25), TGFβ1 (SEQ ID NO: 26), TGFβ2 (SEQ ID NO: 27), TGFβ3 (SEQ ID NO: 28), TREM1 (SEQ ID NO: 29), sTREM2 (SEQ ID NO: 5), CD2AP (SEQ ID NO: 31), FPR2 (SEQ ID NO: 32), P2RY2 (SEQ ID NO: 33), P2RY6 (SEQ ID NO: 34), CHEMR23 (SEQ ID NO: 35), ERV3-2 (SEQ ID NO: 36), HERV-W (SEQ ID NO: 37), HERV-K (SEQ ID NO: 38), GPR18 (SEQ ID NO: 39), GPR32 (SEQ ID NO: 40), GPR37 (SEQ ID NO: 41), or LGR6 (SEQ ID NO: 42.

18. The myeloid cell or mesenchymal cell of claim 9, wherein the transgene is inducible.

19. The myeloid cell or mesenchymal cell of claim 9, wherein the transgene encodes a gene of an inducible gene system, wherein the inducible gene system is a doxorubicin or FK506 binding protein 12 (FKBP) destabilizing domain or protease inducible system.

20. The myeloid cell or mesenchymal cell of claim 15, wherein the vector comprises an additional transgene, wherein the additional transgene encodes IL-IRA (SEQ ID NO: 18), IL-2 (SEQ ID NO: 19), IL-10 (SEQ ID NO: 20), IL-12A (SEQ ID NO: 21), IL-12B (SEQ ID NO: 22), CXCL9 (SEQ ID NO: 23), CXCL10 (SEQ ID NO: 24), SMAD4 (SEQ ID NO: 25), TGFβ1 (SEQ ID NO: 26), TGFβ2 (SEQ ID NO: 27), TGFβ3 (SEQ ID NO: 28), TREM1 (SEQ ID NO: 29), sTREM2 (SEQ ID NO: 5), CD2AP (SEQ ID NO: 31), FPR2 (SEQ ID NO: 32), P2RY2 (SEQ ID NO: 33), P2RY6 (SEQ ID NO: 34, CHEMR23 (SEQ ID NO: 35), ERV3-2 (SEQ ID NO: 36), HERV-W (SEQ ID NO: 37), HERV-K (SEQ ID NO: 38), GPR18 (SEQ ID NO: 39), GPR32 (SEQ ID NO: 40), GPR37 (SEQ ID NO: 41), or LGR6 (SEQ ID NO: 42).

21. The myeloid cell or mesenchymal cell of claim 6, wherein the vector comprises a reporter gene.

22. The myeloid cell or mesenchymal cell of claim 21, wherein the reporter gene is EGFR, tEGFR, or CD90.1.

23-26. (canceled)

27. A myeloid cell or mesenchymal cell comprising exogenous mRNA, wherein the exogenous mRNA encodes viral accessory protein x (Vpx).

28-38. (canceled)

39. A myeloid cell or mesenchymal cell, wherein the cell has been genetically modified to inactivate, knockdown, or remove S100A8 (SEQ ID NO: 44), S100A9 (SEQ ID NO: 45), ARG1 (SEQ ID NO: 46), IDO1 (SEQ ID NO: 47), IL4 (SEQ ID NO: 48), TGFβ1 (SEQ ID NO: 49), TGFβ2 (SEQ ID NO: 50), TGFβ3 (SEQ ID NO: 51), ADAM17 (SEQ ID NO: 52), CD39 (SEQ ID NO: 53), CD73 (SEQ ID NO: 54), CD274, CYBB/gp91phox/NOX2 (SEQ ID NO: 55), BACE1 (SEQ ID NO: 56), NCKAP1L (SEQ ID NO: 57), TREM2 (SEQ ID NO: 30), EP2 (SEQ ID NO: 58), IL12A (SEQ ID NO: 21), IL12B (SEQ ID NO: 22), or TNFA.

40-49. (canceled)

50. A nucleotide sequence for a 3′ UTR that improves the stability of a mRNA sequence, wherein cDNA is used to create the 3′ UTR sequence, and the cDNA comprises the sequence of KJ1 (SEQ ID NO: 6), mtRNR1-KJ1 (SEQ ID NO: 7), mtRNR1-AES-KJ1 (SEQ ID NO: 8), KJ2 (SEQ ID NO: 9), mtRNR1-KJ2 (SEQ ID NO: 10), mtRNR1-AES-KJ2 (SEQ ID NO: 11), or KJ3 (SEQ ID NO: 12).

51. A method of treating a mammal having cancer, a tumor, a neurodegenerative condition, an autoimmune disorder, or an inflammatory disorder, the method comprising administering the myeloid cell or mesenchymal cell of claim 1 to the mammal.
